(19) United States
(12) Patent Application Publication
(10) Pub. No.: US 2009/0085260 A1
(43) Pub. Date: Apr. 2, 2009

(54) BIODEGRADABLE MATERIAL AND PROCESS FOR PRODUCING THE SAME

(75) Inventors: Naotsugu NAGASAWA, Gunma (JP); Toshiaki Yagi, Gunma (JP); Fumio Yoshii, Gunma (JP); Shin-ichi Kanazawa, Osaka (JP); Kiyoshi Kawano, Osaka (JP); Yoshihiro Nakatani, Osaka (JP); Hiroshi Mitomo, Gunma (JP)

Correspondence Address: MCDERMOTT WILL & EMERY LLP, 600 13TH STREET, N.W., WASHINGTON, DC, 20005-3096, US

(73) Assignees: JAPAN ATOMIC ENERGY RESEARCH INSTITUTE, Chiba (JP); SUMITOMO ELECTRIC FINE POLYMER INC, Osaka (JP)

(21) Appl. No.: 12/276,711

(22) Filed: Nov. 24, 2008

Related U.S. Application Data
(62) Division of application No. 10/569,966, filed on Feb. 28, 2006.

(30) Foreign Application Priority Data
Oct. 24, 2003 (JP) 2003-364831
Oct. 24, 2003 (JP) 2003-364892
Oct. 24, 2003 (JP) 2003-364926
Oct. 24, 2003 (JP) 2003-365058

(51) Int. Cl. H05B 6/00; C08L 5/00; C08L 1/12; C08L 3/06; C08L 67/00; C07H 3/00

(52) U.S. Cl. 264/488; 525/54.2; 525/54.21; 525/54.24; 522/134; 536/102; 536/56; 536/123.12; 536/48; 536/32

(57) ABSTRACT

A biodegradable aliphatic polyester, such as polylactic acid, is mixed with a monomer having allyl and molded into a molding having the crosslinking degree of the biodegradable aliphatic polyester increased. Thereafter, the molding is exposed to ionizing radiation to thereby obtain a molding excelling in heat resistance. Triallyl isocyanurate or triallyl cyanurate is used as the monomer having allyl.

### TECHNICAL FIELD

The present invention relates to a biodegradable material and a method for manufacturing the biodegradable material. More particularly, the present invention relates to a biodegradable material made of a synthetic biodegradable polymeric material and excellent in its heat resistance, configuration-retaining property (that is, high hardness), strength, and moldability and to a biodegradable material which has a high heat shrinkage factor and can be used as a heat-shrinkable material and a method for manufacturing the biodegradable material.

### BACKGROUND ART

Many kinds of products such as a film, a container, a heat-shrinkable material, and the like are formed by molding a petroleum synthetic polymer material. But a problem occurs in discarding wastes by burning them after use. That is, social problems have occurred in global warming owing to heat and exhaust gases generated when the products are burnt; in the influence of poisonous substances contained in burnt gases and residues after they are burnt on food and health; and in how to secure places for discarding or embedding the wastes.

To these problems, attention has been paid to a biodegradable polymer represented by starch and polylactic acid as materials that solve the problem of discarding the petroleum synthetic polymer. The biodegradable polymer generates a smaller amount of heat than the petroleum synthetic polymer when the biodegradable polymer is burnt and keeps the cycle of decomposition and re-synthesis in natural environment, thus not giving a bad influence on the global environment including an ecosystem. Above all, aliphatic polyester resin having a characteristic equivalent to the petroleum synthetic polymer in terms of strength and processability is a material to which attention is paid in recent years.

Especially, the polylactic acid is made from starch supplied from plants. Owing to reduction of cost caused by mass production in recent years, the polylactic acid is becoming less expensive than other biodegradable polymers. Thus investigations are now made for its application.

Because the polylactic acid has processability and strength equivalent to general-purpose petroleum synthetic polymer in terms of its characteristic, the polylactic acid is a biodegradable resin closest to a substitute material of the petroleum synthetic polymer. Further because the polylactic acid has a degree of transparency equivalent to that of acrylic resin, the polylactic acid is expected to be used as a substitution thereof. Further because the polylactic acid has a high Young's modulus and a high configuration-retaining property (that is, high hardness), the polylactic acid is expected to be used as a substitution of ABS resin which is used as casings for electric apparatuses and applied to various uses.

However, the polylactic acid has a glass transition point at a comparatively low temperature proximate to 60° C. Young's modulus decreases sharply in the neighborhood of 60° C. to such an extent that as it were, a glass plate suddenly becomes a table cloth made of vinyl resin. Consequently the polylactic acid has a fatal defect that it is difficult for the polylactic acid to hold its shape which the polylactic acid has at a low temperature.

The crystalline portion of the polylactic acid which does not melt until it reaches a melting point of 160° C. is a crystallite not showing a large mass. The polylactic acid is not so structured that only the crystalline portion supports the entire strength at a normal crystallinity. This is a cause of a rapid change of the Young's modulus. The rapid change of the Young's modulus occurs in the vicinity of the glass transition point at which a non-crystalline portion moves freely. Thus the rapid change of the Young's modulus is mainly attributed to the fact that the non-crystalline portion almost loses an interaction in molecules at not less than 60° C.

It is known that to improve heat resistance, a material is irradiated with radioactive rays to introduce a crosslinked structure thereinto. For example, it is known that heat-resistant polyethylene is obtained by irradiating polyethylene, used as general-purpose resin, which melts in the neighborhood of 100° C. with radioactive rays of about 100 kGy. It is also known that when a reactive polyfunctional monomer is added to a material consisting of a polymer that is liable to decompose and to a material having low crosslinking efficiency, crosslinking can be accelerated by the irradiation of the radioactive rays thereto.

In adding the polyfunctional polymer to the biodegradable polymer, normally the polyfunctional monomer is added thereto at a high concentration of not less than 5 wt % of the whole weight. When the biodegradable material to which the polyfunctional monomer has been added at a high concentration is irradiated with the radioactive rays, it is difficult to react them at 100% and thus unreacted monomer remains. Thereby a problem occurs that the crosslinking efficiency is low, and the biodegradable material is deformed easily by heating and has a deteriorated heat resistance.

Normally not less than 99% of the biodegradable material is classified as being decomposed by the action of microorganisms. Thus when a crosslinking technique using the polyfunctional monomer is applied to the biodegradable material, the biodegradable material does not fall under the category of the biodegradable material in dependence on a concentration of the polyfunctional monomer.

Regarding the improvement of the heat resistance of the biodegradable polymer, it is known that the polylactic acid is only decomposed when it is irradiated with the radioactive rays and that effective crosslinking cannot be obtained.

As the biodegradable material in medical use, in Japanese Patent Application Laid-Open No. 2002-114921 (patent document 1) and Japanese Patent Application Laid-Open No. 2003-695 (patent document 2), disclosure is made on the irradiation of the radioactive rays which is performed not for the improvement of the heat resistance but for sterilization.

That is, provided in the patent document 1 is the composition in which the decrease of the weight-average molecular weight after molding by heating the biodegradable polymer and performing sterilization by radiant ryas is suppressed to not more than 30% of the initial weight-average molecular weight by adding a polyfunctional monomer such as triallyl isocyanurate to the biodegradable polymer.

Provided in the patent document 2 is the material for medical use composed of the polymeric substances such as collagen, gelatin, polylactic acid, and polycaprolactam which contain the polyfunctional triazine compound such as triallyl isocyanurate. The material for medical use can be sterilized by irradiating it with radioactive rays.

The compositions disclosed in the patent documents 1 and 2 contain the polyfunctional monomers to suppress a decrease of the molecular weight of the biodegradable polymer in heat history at the time of molding by heating the biodegradable polymer and in the sterilization process by means of the irradiation of radioactive rays.

It is disclosed in the patent document 1 that the addition amount of the free radical scavenger is preferably not less than 0.01 wt % for 100 wt % of the biodegradable polymer and that in the examples, as the free radical scavenger, 0.2 wt % of the triallyl isocyanurate is added to 100 wt % of the polylactic acid, and the mixture is irradiated with γ-rays at 20 kGy.

However, according to additional tests made by the present inventors, it has been found that when the addition amount of the triallyl isocyanurate is 0.2 wt %, a crosslinking reaction hardly occurs and the gel fraction percentage is less than 3%, even though the biodegradable polymer is irradiated with γ-rays of 20 kGy. Therefore the biodegradable polymer has hardly a crosslinked structure and thus cannot be provided with heat resistance.

It is described in the patent document 2 that not less than 0.01 wt % of the polyfunctional triazine compound consisting of the triallyl isocyanurate is added to the biodegradable polymer and that in the examples, 1 wt % of the triallyl isocyanurate is added to the polylactic acid, the polylactic acid is irradiated with γ-rays at 25 kGy, and the gel fraction percentage thereof is set to 67%. However, when the gel fraction percentage is 67%, the polylactic acid is liable to deform in an atmosphere having a high temperature exceeding 60° C. which is the glass transition temperature of the polylactic acid. Thus improvement is not made for the polylactic acid which is low in its configuration-retaining property (that is, high hardness) and inferior in its heat resistance.

As a method for making the polylactic acid heat-resistant, the following technique is disclosed in “Grade advanced.terramack of injection molding of highly heat-resistant polylactic acid” described in a magazine “Plastic Age” (not in patent document 1): The mineral filler of nano-order fine particles is mixed with the polylactic acid to increase the crystallinity in a comparatively short period of time with the particles serving as the nucleus. The method described in the above-described thesis makes it possible to take a mixture thereof from a die in an order of several tens of minutes to several minutes, thus allowing the heat-resistant polylactic acid to be manufactured. Although improvement is made in terms of the cost in an industrial production, not less than 1 to 5 wt % of the untransparent clay filler is added to the whole weight of the polylactic acid. Therefore the polylactic acid loses the transparency thereof. Further the filler roughens the surface of the polylactic acid which is originally glossy like glass. Thus the product composed of the composition has defects that it looks not fine and hence products composed of the composition can be utilized in a limited range.

Further it is impossible to disperse the mineral filler, added to the polylactic acid, in a size larger than the original size thereof. Thus a variation is liable to occur in the strength of the composition. Further there is no fundamental bonding between the mineral filler and the base consisting of resin, and the reinforcing effect depends on the strength of the filler itself. Thus it is necessary to increase the addition amount of the filler to enhance the strength of the composition. But the increase of the addition amount of the filler deteriorates the above-described transparency and smoothness. Another problem is that when the mixture containing the filler is molded, a breeding phenomenon that the filler comes out of the resin that is the base of the composition is liable to occur with time.

As a method of improving the disadvantage that the polylactic acid does not have the configuration-retaining property (that is, high hardness) at a high temperature and is inferior in its heat resistance, by decreasing the non-crystalline portion of the polylactic acid and increasing the crystallinity thereof to 90 to 95%, it is possible to prevent the polylactic acid from softening at temperatures not less than 60° C. and maintain the shape thereof.

However, as the method of increasing the crystallinity of the polylactic acid, it is necessary to mold the polylactic acid into various shapes by melting it by injection molding or the like and thereafter wait for a long time until crystallization progresses at a temperature not less than the glass transition temperature nor more than the fusing temperature thereof. Thus for example, to produce a component part having a thickness in the range from several millimeters to a little less than one centimeter, it is necessary to hold the part in a die while it is being heated for several tens of minutes after injection molding finishes. Thus this method cannot be utilized in an industrial production and is thus unrealistic.

Regarding the case in which the biodegradable material is used as a heat-shrinkable material, a convenient heat-shrinkable material that can shrink at a temperature not less than 100 to 120° C. and at a shrinkage rate not less than 40% has not been provided.

As the heat-shrinkable material made of this kind of the biodegradable material, in Japanese Patent Application Laid-Open No. 2003-221499 (patent document 3), there is disclosed the polylactic acid-based heat-shrinkable material whose transparency is improved by adding polycarbodiimide to the mixture of the polylactic acid-based polymer and aliphatic polyester other than the polylactic acid-based polymer.

However, in the heat-shrinkable material containing the polylactic acid, the glass transition temperature of the polylactic acid is 50° C. to 60° C. Therefore the polylactic acid-based heat-shrinkable material is deformable and inferior in its heat resistance. The polylactic acid-based heat-shrinkable material disclosed in the patent document 3 expands at 70 to 80° C. a little higher than the glass transition temperature (a little less than 60° C.) of the polylactic acid when it is heated, and does not expand at not less than the melting point of the polylactic acid. Thus it shrinks thermally at its crystalline portion which has a low restoring force against deformation. Thus the heat shrinkage factor of the polylactic acid-based heat-shrinkable material is only 30 to 40%.

Cellulose and starch are materials hydrophilic with water. Thus when the cellulose and the starch get wet, it is difficult for the cellulose and the starch to keep its strength unlike a petroleum synthetic polymer substance. Further the cellulose and the starch cannot be molded by melting them, unlike the petroleum synthetic polymer having a clear melting point. To mold the starch, after it is molded in a melted state like a liquid containing water, it is necessary to remove the water by drying it as necessary. The starch mixed with the water is flexible, but has a very low strength. On the other hand, dried starch is frail and lack flexibility.

This characteristic is attributed to the hydroxyl group of the cellulose and the starch. That is, the hydroxyl group shows hydrophilic property owing to its strong polarizing property, and further a strong hydrogen bonding is formed between hydroxyl groups and this bonding is stable against heat. To mold the starch like the petroleum synthetic polymer by heating the starch to melt it, the starch derivative obtained by modifying the hydroxyl group of the starch by esterification and making the esterified hydroxyl group hydrophobic is disclosed in U.S. Pat. Nos. 2,579,843 and 3,154,056.

However, the esterified starch derivative made hydrophobic is hardly elastic and frail. For example, in the esterification made by using the above-described fatty acid, acetate ester starch using the above-described fatty acid having the lowest molecular weight as the fatty acid of substituting group has a certain degree of strength but is hardly elastic and has a very high Young's modulus and is thus a very frail resin having glass-like property.

When a fatty acid having a high molecular weight, namely, a higher fatty acid is used for esterification, intermolecular forces decrease in molecules of starch. Consequently the starch derivative becomes deformable and can be made elastic. But as a result of the decrease in the intermolecular forces, the strength thereof decreases.

Products made of the hydrophobic starch derivative commercially available are improved in its strength and expansion percentage by adding biodegradable polyester or the mineral filler to the hydrophobic starch derivative, as disclosed in Japanese Patent Application Laid-Open No. 8-502552 (patent document 4).

However, the addition of the biodegradable polyester to the hydrophobic starch does not improve the strength characteristic of the hydrophobic starch itself, but merely allows the hydrophobic starch to approach the characteristic of the biodegradable polyester mixed therewith and needless to say, and makes the hydrophobic starch derivative inferior in its strength to the biodegradable polyester added thereto. Thus there is a doubt in the necessity of using the expensive hydrophobic starch. Further the addition of the mineral filler to the hydrophobic starch derivative damages the smoothness and transparency. Thus there is a limitation in the use of the product containing the mineral filler.

It is known that to enhance the strength, radioactive rays are irradiated to a material to allow it to have a crosslinked structure. However, starch and cellulose of natural decomposable polysaccharides and derivatives thereof are substances that are decomposed by being irradiated with radioactive rays. Thus they are decomposed when they are subjected to the radioactive rays. Regarding the crosslinking of the derivatives of the starch and the cellulose performed by the radioactive rays, it is known that a crosslinked material is obtained by irradiating a high-concentration mixture of water and the above derivatives with the radioactive rays after the mixture is heated. That is, water is essential for the crosslinking to be performed by means of the radioactive rays. Even in the case where a chemical bonding is performed without using the radioactive rays, a reaction in a system not containing water is hardly made.

Because the derivative of the hydrophobic starch is not soluble in water, it cannot be kneaded with water. Therefore the hydrophobic cannot be crosslinked by using a conventional radioactive rays-using crosslinking art. Further the hydrophobic cannot be crosslinked by means of a crosslinking agent such as aldehyde for use in chemical treatment in crosslinking the starch.

Patent document 1: Japanese Patent Application Laid-Open No. 2002-114921

Patent document 2: Japanese Patent Application Laid-Open No. 2003-695

Patent document 3: Japanese Patent Application Laid-Open No. 2003-221499

Patent document 4: Japanese Patent Application Laid-Open No. 8-502552

Non-patent document 1: “Grade advanced.terramack of injection molding of highly heat-resistant polylactic acid” (the April, 2003, issue of “Plastic Age”, pages 132 to 135)

### DISCLOSURE OF THE INVENTION

### Problem to be Solved by the Invention

The present invention has been made in view of the above-described problems. Therefore it is an object of the present invention to provide a biodegradable material which is improved in its heat resistance to use products composed as the biodegradable material as substitutions of products, made of molded plastics, such as a film, a packing material, a protecting material, a sealing material, and the like, and which is capable of solving the problem of discarding wastes after use owing to its biodegradable performance, and provide a manufacturing method which is industrially practical.

More specifically, a first object is to provide a biodegradable material which is allowed to be heat-resistant by improving configuration-retaining property (that is, high hardness) which deteriorates at not less than the glass transition point and which is not damaged in its transparency, glossiness of its surface, and smoothness.

A second object is to provide a biodegradable material that has a high heat shrink characteristic, can be preferably used in environment having a high temperature, and can be used as a heat-shrinkable material.

A third object is to provide a biodegradable material having both a strength and an expansion to such an extent that the biodegradable material can be used as a substitution of the petroleum synthetic polymer without adding large amounts of other substances to a hydrophobic polysaccharide derivative.

### Means for Achieving the Objects

To achieve the first object of enhancing the heat resistance of the biodegradable material, the present inventors have made energetic researches and found that the object can be achieved by mixing a monomer having an allyl group with biodegradable aliphatic polyester and by irradiating the mixture with radioactive rays to crosslink molecules to each other satisfying predetermined conditions. Particularly, the present inventors have found that the configuration-retaining property (that is, high hardness) of the polylactic acid at a high temperature can be improved by sufficiently crosslinking the non-crystalline portion of the polylactic acid with the allyl group-containing monomer, although the polylactic acid is decayed by radioactive rays and is conventionally considered non-crosslinkable with ordinary monomers.

Based on the above-described finding, the first invention provides a biodegradable material which contains biodegradable aliphatic polyester at not less than 95 wt % nor more than 99 wt % of a whole weight thereof and has a crosslinked structure in such a way that the biodegradable aliphatic polyester has a gel fraction percentage (gel fraction dried weight/initial dried weight) not less than 75% nor more than 95 to allow the biodegradable material to be heat-resistant.

To measure the gel fraction percentage, a predetermined amount of a film is enclosed in a metal net of 200 meshes, it is boiled in a solvent of chloroform for 48 hours, a dissolved sol content is removed, and a gel fraction which has remained on the metal net is dried. In this manner, the weight of the film is found. The gel fraction percentage is computed by an equation shown below:

gel fraction percentage (%)=(gel fraction dried weight)/(initial dried weight)×100

As described above, in the biodegradable material of the first invention, the gel fraction percentage of the polymer consisting of the biodegradable aliphatic polyester which is the main component of the biodegradable aliphatic polyester is set to not less than 75%, and more than 75% of the polymer is formed as the crosslinked structure to generate innumerable three-dimensional screen structures in the polymer. Thus it is possible to allow the biodegradable material to have heat resistance to such an extent that it does not deform at temperatures not less than the glass transition temperature of the polymer. Therefore the biodegradable material has improved heat resistance, has configuration-retaining property (that is, high hardness) similar to that of resin products made of the petroleum synthetic polymer, and thus can be utilized as substitutions thereof. In addition, since the biodegradable material of the present invention is biodegradable, the present invention is capable of solving the problem of discarding wastes.

As the method for manufacturing the heat-resistant biodegradable material of the first invention having the crosslinked structure, it is preferable that 1.2 wt % to 5 wt % of the monomer having the allyl group and 100 wt % of the biodegradable aliphatic polyester are kneaded; an obtained uniform mixture is pressed by heating the uniform mixture under pressure and then cooled rapidly to mold the uniform mixture into a required shape; and the molded uniform mixture is irradiated with ionizing radiation to generate a crosslinking reaction in such a way that the biodegradable aliphatic polyester is crosslinked at not less than 75% of the whole weight thereof.

It is preferable to use the polylactic acid as the biodegradable aliphatic polyester and use triallyl isocyanurate or triallyl cyanurate as the monomer having the allyl group.

That is, an object of the present invention is to provide the biodegradable material which has various properties equivalent to those of the general-purpose petroleum synthetic polymer and is capable of substituting it. Therefore as the biodegradable aliphatic polyester that is used for achieving the object of the present invention, the polylactic acid, isomers L- and D- thereof, and a mixture thereof, polybutylene succinate, polycaprolactam, and polyhydroxy butyrate are listed. These substances can be used singly or in combination of two or more thereof. In terms of the cost and characteristics, polylactic acids are especially suitable.

To improve the flexibility of the biodegradable material, as additives which can be added thereto, it is possible to use a plasticizer such as glycerin, ethylene glycol, triacetyl glycerin liquid at the normal temperature or biodegradable resin which is used as a plasticizer such as polyglycolic acid and polyvinyl alcohol solid at the normal temperature. In addition, it is possible to add a small amount of other biodegradable aliphatic polyesters as a plasticizer to the polylactic acid. But the use of these plasticizers is not essential in the present invention.

As monomers to be mixed with the aliphatic polyester, the following acrylic monomers or methacrylic monomers having two or more double bonds in one molecule are effective: 1,6-hexanediol diacrylate, trimethylolpropane trimethacrylate (hereinafter referred to as TMPT), and the like. But to obtain a high degree of crosslinking at a comparatively low concentration, the following monomers having an allyl group are effective.

Triallyl isocyanurate, trimethallyl isocyanurate, triallyl cyanurate, trimethallyl cyanurate, diallyl amine, triallyl amine, diacryl chlorendate, allyl acetate, allyl benzoate, allyl dipropyl isocyanurate, allyl octyl oxalate, allyl propyl phthalate, butyl allyl malate, diallyl adipate, diallyl carbonate, diallyl dimethyl ammonium chloride, diallyl fumarate, diallyl isophthalate, diallyl malonate, diallyl oxalate, diallyl phthalate, diallyl propyl isocyanurate, diallyl sebacate, diallyl succinate, diallyl terephthalate, diallyl tatolate, dimethyl allyl phthalate, ethyl allyl malate, methyl allyl fumarate, and methyl methallyl malate.

Of these monomers, the triallyl isocyanurate (hereinafter referred to as TAIC) and the trimethallyl isocyanurate (hereinafter referred to as TMAIC) are especially desirable. The TAIC is particularly effective for the polylactic acid. The triallyl cyanurate and the trimethallyl cyanurate convertible with the TAIC and the TMAIC respectively by heating are substantially similar to the TAIC and the TMAIC in the effect thereof.

As the above-described ionizing radiation, γ-rays, x-rays, β-rays or α-rays can be used. In industrial production, γ-rays emitted from cobalt 60 and electron beams emitted from an electron accelerator are preferable. To introduce the crosslinked structure, the ionizing radiation is irradiated. But a chemical initiator may be used to generate a crosslinking reaction.

In this case, the monomer having the allyl group and the chemical initiator are added to the biodegradable material at a temperature not less than the melting point of the biodegradable material, and they are kneaded sufficiently and mixed with one another uniformly. Thereafter a molded material is heated to a temperature at which the chemical initiator is thermally decomposed.

As the chemical initiator which can be used in the present invention, it is possible to use any of peroxide catalysts generating peroxidic radicals such as dicumyl peroxide, propionitrile peroxide, penzoil peroxide, di-t-butyl peroxide, diasyl peroxide, beralgonyl peroxide, mirystoil peroxide, tert-butyl perbenzoate, and 2,2′-azobisisobutyronitrile; and catalysts for starting polymerization of monomers. Similarly to the irradiation of radioactive rays, it is preferable to perform crosslinking in an air-removed inert atmosphere or in a vacuum.

To achieve the above-described first object, the present inventors have made energetic researches and found that the object can be achieved by integrating biodegradable aliphatic polyester and a hydrophobic polysaccharide derivative with each other by means of crosslinking.

The above-described integration means that in a solvent in which two components are singly soluble, at least one part of both components is contained in the solvent as a component of a substance made insoluble owing to crosslinking.

The biodegradable material of the second invention which has been completed based on the above-described knowledge consists of a heat-resistant crosslinked material composed of the biodegradable aliphatic polyester and the hydrophobic polysaccharide derivative integrated therewith by means of crosslinking.

As the method of the second invention for manufacturing the heat-resistant biodegradable material, after the biodegradable aliphatic polyester, the hydrophobic polysaccharide derivative, and the polyfunctional monomer are mixed uniformly with one another at a temperature not less than the melting point of the biodegradable aliphatic polyester, the mixture is irradiated with ionizing radiation.

In the heat-resistant biodegradable material of the second invention having the crosslinked structure, the biodegradable aliphatic polyester is crosslinked with the hydrophobic polysaccharide derivative to integrate them with each other and form innumerable three-dimensional network structures in the polymer. Thus it is possible to allow the biodegradable material to have heat resistance to such an extent that it does not deform at temperatures not less than the glass transition temperature of the polymer. Particularly, when a substantial fusion molding temperature is set to the range of 150° C. to 200° C. which is not less than the melting point of the biodegradable aliphatic polyester and not less than the softening point of the hydrophobic polysaccharide derivative, the tensile strength of the biodegradable material and the expansion percentage thereof at high temperatures in the vicinity of 150° C. to 200° C. are 30 to 70 g/mm² and 20 to 50% respectively. That is, the biodegradable material is set low in its expansion percentage and high in its tensile strength.

As described above, because the biodegradable material is set low in its expansion percentage and high in its tensile strength at high temperatures to prevent its deformation, the biodegradable material has a configuration-retaining property (that is, high hardness) at high temperatures, and the heat resistance which is the disadvantage of the biodegradable material is improved. Thus the biodegradable material can be widely used as materials for industrial products. Therefore the products made of the biodegradable material has a configuration-retaining property (that is, high hardness) similar to that of general-purpose resin products made of the petroleum synthetic polymer and can be utilized as substitutions thereof. Further because the products made of the biodegradable material is biodegradable, the problem of discarding wastes can be solved by the use of the products made of the biodegradable material.

In the heat-resistant biodegradable material of the second invention having the crosslinked structure, as the biodegradable aliphatic polyester, polylactic acid similar to that of the first invention is used. As the crosslinking-type polyfunctional monomer, the monomer having the allyl group similar to that of the first invention is preferably used. As the ionizing radiation, radioactive rays similar to that of the first invention are preferably used. Instead of the ionizing radiation, a chemical initiator may be used to generate the crosslinking reaction.

To achieve the second object, the third invention provides a biodegradable material whose heat shrinkage factor can be made high and which can be used as a heat-shrinkable material.

The heat-shrinkable biodegradable material of the third invention is composed of a mixture of biodegradable aliphatic polyester and a low-concentration monomer having an allyl group. In a state in which the mixture is crosslinked by irradiating the mixture with ionizing radiation or adding a chemical initiator to the mixture, the mixture is expanded with heat being applied thereto. When the mixture is heated at a temperature not less than a temperature used at an expanding time, a shrinkage factor of the mixture is not less than 40% nor more than 80%.

More specifically, the polylactic acid is used as the biodegradable aliphatic polyester. The gel fraction percentage (gel fraction dried weight/initial dried weight) obtained by crosslinking the mixture is set to 10 to 90%. The shrinkage factor of the mixture is set less than 10% at not more than 140° C. and 40 to 80% at not less than 160° C.

The above-described heat shrinkage factor is defined as follows:

In the case of a sheet:

(length) shrinkage factor (%)=(length before shrinkage−length after shrinkage)/(length before shrinkage)×100, and

In the case of a tube:

(inner diameter) shrinkage factor (%)=(inner diameter before shrinkage−inner diameter after shrinkage)/(inner diameter before shrinkage)×100.

Therefore when the shrinkage factor is 50%, the length of the sheet (tube) becomes ½ (50%) of the original length (inner diameter).

When the shrinkage factor is 80%, the length of the sheet (tube) becomes 20% of the original length (inner diameter).

As described above, in the third invention, the addition amount of the crosslinking-type polyfunctional monomer is set to a range in which the mixture is gelled to some extent, and further a possible smallest amount of the crosslinking-type polyfunctional monomer is used to make the concentration thereof low. Thereby the gel fraction percentage is set to 10 to 90% and preferably 50 to 70% when the mixture is irradiated with the ionizing radiation in a subsequent step so that the heat resistance of the biodegradable material and the shrinkage factor thereof are enhanced. When the gel fraction percentage is too low, needless to say, a network to be stored is not formed and shrinkage does not occur.

The gel fraction percentage required for the conventional heat-shrinkable material composed of the petroleum synthetic resin to shrink is 10 to 30%, whereas in the present invention, the biodegradable material is allowed to be heat-shrinkable when the gel fraction percentage of the aliphatic polyester, particularly the gel fraction percentage of the polylactic acid is increased to 90%.

When the gel fraction percentage is too high, the network formed by the crosslinking is so firm that the deformation amount, namely, the elongative amount is small, although a shrinkable force is high. As a result, the shrinkage factor is low. Thus the gel fraction percentage is preferably in the range of 50 to 70%.

In the method of the third invention for manufacturing the heat-shrinkable biodegradable material, the crosslinking-type polyfunctional monomer is added at a low concentration to the biodegradable material, and the crosslinking-type polyfunctional monomer and the biodegradable material are kneaded; and the mixture is molded into a predetermined shape by heating the mixture under pressure, and the mixture is cooled rapidly; the mixture is irradiated with ionizing radiation to generate a crosslinking reaction so that a gel fraction percentage thereof is set to not less than 10% nor more than 90%; and after the mixture is irradiated with the ionizing radiation, the mixture is expanded while the mixture is being heated at a temperature not less than a fusing temperature of the biodegradable material nor more than a temperature obtained by an addition of the fusing temperature and 20° C. to form the mixture as a heat-shrinkable material.

In the heat-shrinkable biodegradable material of the third invention having the crosslinked structure, as the biodegradable aliphatic polyester, polylactic acid similar to that of the first invention is used. As the crosslinking-type polyfunctional monomer, the monomer having the allyl group similar to that of the first invention is preferably used. As the ionizing radiation, radioactive rays similar to that of the first invention are preferably used. Instead of the ionizing radiation, a chemical initiator may be used to generate the crosslinking reaction.

To achieve the third object, the present inventors have made energetic researches and found that it is possible to accomplish the crosslinking by means of radioactive rays by kneading the polyfunctional monomer with the hydrophobic polysaccharide derivative and then irradiating the mixture with the ionizing radiation and that the hydrophobic polysaccharide derivative such as acetate ester starch and cellulose crosslinked by using the radioactive rays are excellent in the strength and expansion thereof.

Based on the above-described finding, the fourth invention provides a biodegradable material composed of the hydrophobic polysaccharide derivative and the crosslinking-type polyfunctional monomer, such as the monomer having the allyl group, added to the hydrophobic polysaccharide derivative to allow a mixture of the hydrophobic polysaccharide derivative and the crosslinking-type polyfunctional monomer to have a crosslinked structure having a gel fraction percentage (gel fraction dried weight/initial dried weight) at 10 to 90%.

In the method of the fourth invention for manufacturing the biodegradable material, the polyfunctional monomer is added to the hydrophobic polysaccharide derivative, a mixture of the polyfunctional monomer and the hydrophobic polysaccharide derivative is kneaded, and after the mixture is molded into a predetermined shape, a molded material is irradiated with ionizing radiation to generate a crosslinking reaction so that the biodegradable material has a crosslinked structure.

In the biodegradable material of the fourth invention, as the crosslinking-type polyfunctional monomer, the monomer having the allyl group similar to that of the first invention is preferably used. As the ionizing radiation, radioactive rays similar to that of the first invention are preferably used. Instead of the ionizing radiation, a chemical initiator may be used to generate the crosslinking reaction.

### EFFECT OF THE INVENTION

As described above, because the biodegradable material of each of the first through fourth inventions have an enhanced heat resistance, they are widely applicable. Especially, the biodegradable material hardly affects an ecosystem adversely in nature. Thus the biodegradable material can be used as a material substituting plastic products mass-produced and discarded. In addition, because the biodegradable material does not give a bad influence on the organism, it is suitably applicable to medical appliances which are used inside and outside the organism.

Because the gel fraction percentage of the heat-resistant biodegradable material of the first invention is set to 75 to 95%, the heat resistance of the biodegradable aliphatic polyester can be greatly improved.

The heat-resistant biodegradable material of the second invention is capable of improving the configuration-retaining property (that is, high hardness) of the biodegradable aliphatic polyester, particularly that of the polylactic acid at temperatures not less than 60° C. Further because the hydrophobic polysaccharide derivative is added to the polylactic acid to maintain the strength of the biodegradable material at high temperatures, the transparency of the polylactic acid and the glossiness of the surface thereof are not damaged greatly unlike the case in which the mineral filler is used. Furthermore although it is necessary to set a high temperature in an industrial production, the biodegradable material can be manufactured by using conventional injection molding equipment without deteriorating productivity. Further because the hydrophobic polysaccharide derivative is biodegradable, it hardly affects an ecosystem adversely in nature. Thus it is expected that the biodegradable material be used as a material which substitutes plastic products mass-produced and discarded.

The heat-shrinkable biodegradable material of the third invention can be expanded to about five times as long as its original length. When the expanded heat-shrinkable material is heated to a temperature not less than its melting point, it can be thermally shrunk at a shrinkage factor of 40 to 80% owing to the network whose shape is stored. Owing to the crystalline portion and the network which do not melt at about the glass transition temperature of the polylactic acid, the heat-shrinkable material does not deform its shape and has heat-resistant property.

The fourth invention has succeeded in crosslinking the hydrophobic polysaccharide derivative by irradiating it with the ionizing radiation. Further a low strength which is the disadvantage of the hydrophobic polysaccharide derivative can be improved greatly by the molecule-crosslinking effect. The effect can be expected particularly at a high temperature. Further because the hydrophobic polysaccharide derivative is also biodegradable, it hardly affects an ecosystem adversely in nature. Thus it is expected that the biodegradable material of the fourth invention be used as a material which substitutes plastic products mass-produced and discarded.

### BRIEF DESCRIPTION OF THE DRAWINGS

FIG. 1 is a graph showing the relationship between an irradiation dose of electron beams and a gel fraction percentage in examples 1 through 5 of a first embodiment of the present invention and comparison examples 1 through 8.

FIG. 2 is a graph showing the relationship between a tensile strength and the irradiation dose of the electron beams in a tensile test conducted in an atmosphere of 180° C. in the examples 1 through 5 of the first embodiment of the present invention and the comparison examples 1 through 8.

FIG. 3 is a graph showing the relationship between a breaking expansion and the irradiation dose of the electron beams in the tensile test conducted in the atmosphere of 180° C. in the examples 1 through 5 of the first embodiment of the present invention and the comparison examples 1 through 8.

FIG. 4 is a graph showing the relationship between an irradiation dose of electron beams and a gel fraction percentage in examples 6 through 11 of a second embodiment of the present invention and comparison examples 9 through 18.

FIG. 5 is a graph showing the relationship between a tensile strength and the irradiation dose of the electron beams in a tensile test conducted in an atmosphere of 100° C. in the examples 6 through 8 of the second embodiment of the present invention and comparison examples 15 and 16.

FIGS. 6(A) through (D) are schematic views showing a crosslinked structure, an expanded structure, a structure at the time of a glass transition temperature, and a heat shrinkage structure of a sheet of a third embodiment of the present invention.

FIGS. 7(A) through (D) are schematic views showing a sheet not crosslinked.

FIG. 8 is a graph showing the relationship between an irradiation dose of electron beams and a gel fraction percentage.

FIG. 9 is a graph showing the relationship between a shrinkage temperature and a shrinkage factor.

FIG. 10 is a graph showing a change of a gel fraction percentage with respect to an irradiation dose of electron beams in examples 12, 13, 18, and 19 of a fourth embodiment of the present invention and a comparison example 27.

FIG. 11 is a graph showing a change of a tensile break strength with respect to the irradiation dose of the electron beams in the example 12 of the fourth embodiment of the present invention and the comparison example 27.

### EXPLANATION OF REFERENCE SYMBOLS AND REFERENCE NUMERALS

A: crystalline portion
B: non-crystalline portion
C: network

### BEST MODE FOR CARRYING OUT THE INVENTION

The biodegradable material of the first embodiment is composed of the heat-resistant crosslinked material of the first invention. Not less than 95 wt % nor more than 99 wt % of the whole weight of the biodegradable material consists of biodegradable aliphatic polyester. The biodegradable material is crosslinked in such a way that the biodegradable aliphatic polyester has a gel fraction percentage (gel fraction dried weight/initial dried weight) not less than 75% nor more than 95%.

To accelerate a crosslinking reaction for the biodegradable aliphatic polyester, 1.2 to 5 wt % of the monomer having the allyl group is mixed with 100 wt % of the biodegradable aliphatic polyester. To accelerate the crosslinking reaction at 3 wt %, 1.2 to 3 wt % is preferable.

As the biodegradable aliphatic polyester, the polylactic acid is used. To improve the flexibility thereof, the above-described plasticizer may be added thereto.

As the monomer to be mixed with the aliphatic polyester, the monomer having the allyl group is effective. As the monomer having the allyl group, triallyl isocyanurate (hereinafter referred to as TAIC) and trimethallyl isocyanurate (hereinafter referred to as TMAIC) can be preferably used.

The crosslinking can take place when not less than 0.5 wt % of the above-described monomer is added to 100 wt % of the biodegradable polymer. But to achieve the object of the present invention of attaining not less than 75% in the gel fraction percentage at which the effect of improving the strength of the biodegradable material can be securely obtained at a high temperature, as the concentration of the monomer, 1.0 wt % is not sufficient but not less than 1.2 wt % is required. However, even if the concentration of the monomer is increased to not less than 3 wt %, there is not much difference in the effect. When the addition amount of the monomer is not less than 5 wt %, there is little difference in the effect. Considering that the biodegradable material is used as biodegradable plastic, it is desirable that the biodegradable material contains much polysaccharide that is securely decomposed. Therefore the addition amount of the monomer is set to the range of 1.2 to 5 wt % and favorably to the range of 1.2 to 3 wt %.

The biodegradable material of the first embodiment is not less than 150° C. nor more than 200° C. in its melting point, 20 to 100 g/mm² in its tensile strength at a high temperature in the vicinity of its melting point, and 30 to 100% in its expansion percentage. That is, the expansion percentage of the biodegradable material is set low, and the tensile strength thereof is set high.

As described above, at a high temperature in the vicinity of its melting point, the expansion percentage of the biodegradable material is set low and the tensile strength thereof is set high to prevent the biodegradable material from being deformed. Thus at the high temperature, the biodegradable material has a configuration-retaining property (that is, high hardness) and is allowed to have an enhanced heat resistance and hence can be widely used for industrial products and articles of practical use.

As the method for manufacturing the biodegradable material of the first invention, 1.2 wt % to 5 wt % of the monomer having the allyl group and 100 wt % of the biodegradable aliphatic polyester are kneaded; the uniform mixture of the biodegradable aliphatic polyester and the monomer is pressed by heating the uniform mixture under pressure and cooled rapidly to mold the uniform mixture into a required shape; and the uniform mixture is irradiated with the ionizing radiation to generate a crosslinking reaction in such a way that the biodegradable aliphatic polyester is crosslinked at not less than 75% of the whole weight of the biodegradable aliphatic polyester.

The irradiation dose of the ionizing radiation depends on the concentration of the monomer to some extent. Although the crosslinking occurs in an amount of even 5 to 10 kGy, the effect of the crosslinking and the effect of improving the strength of the biodegradable material at the high temperature can be obtained at not less than 20 kGy and more desirably not less than 30 kGy at which the effect can be securely obtained. The polylactic acid preferable as the aliphatic polyester will be decayed by itself with radioactive rays. Thus when the irradiation dose is more than the necessary irradiation dose, the decomposition of the polylactic acid will progress in reverse to the crosslinking. Accordingly the irradiation dose is set to not more than 150 kGy and favorably not more than 100 kGy. More favorably the irradiation dose is set to 20 kGy to 50 kGy.

More specifically, the aliphatic polyester is heated to a temperature at which it is softened by heating or dissolved and dispersed in a solvent dissolvable in chloroform, cresol or the like. Thereafter the monomer having the allyl group is added thereto, and these substances are mixed with each other as uniformly as possible. Thereafter the mixture is heated to soften it so that it is molded into a desired shape. The molding may be performed in the state in which the mixture is softened by heating or in the state in which the mixture is dissolved in the solvent. Alternatively the mixture may be molded into a desired shape by injection molding or the like by heating it again to soften it after it is cooled or the solvent is removed by drying it.

Thereafter the molded material is irradiated with the ionizing radiation to generate the crosslinking reaction.

Although the molded material is irradiated with the ionizing radiation to allow it to have the crosslinked structure, the above-described chemical initiator may be mixed with the aliphatic polyester to generate the crosslinking reaction.

In this embodiment, 1.2 to 5 wt % of the TAIC (triallyl isocyanurate) is added to 100 wt % of the polylactic acid dissolved in the solvent. Thereafter a mixture is kneaded, molded by heating (thermal press) it at 180° C. under pressure, and thereafter cooled rapidly at about 100° C./minute to the normal temperature. Thereby a sheet having a required thickness is obtained.

In an air-removed inactive atmosphere, the sheet is irradiated with electron beams at an irradiation dose of 20 to 100 kGy, an applied voltage of 2 MeV, and a current value of 1 mA to progress the crosslinking of molecules of the polylactic acid by means of the TAIC. After the crosslinking finishes, the gel fraction percentage is 75% to 95%.

The heat-resistant crosslinked material is set to 20 to 100 g/mm² in its tensile strength and 30 to 100% in its expansion percentage at a high temperature of 180° C. higher than 160° C. which is the melting point of the polylactic acid. That is, the expansion percentage of the heat-resistant crosslinked material is set low and the tensile strength thereof is set high at high temperature to make the configuration-retaining property (that is, high hardness) thereof high.

### EXAMPLE 1

As the aliphatic polyester, finely powdered polylactic acid (Racia H-100J manufactured by Mitsui Kagaku) was used. 1.2 wt % of the TAIC (manufactured by Nippon Kasei Inc.) which is the allyl monomer was added to the polylactic acid which was melted at 180° C. by using a Lab Plast mill which is a substantially closed kneader, and sufficiently kneaded until it became transparent. The mixture was sufficiently kneaded at 20 rpm for 10 minutes. Thereafter the uniform mixture was thermally pressed at 180° C. to obtain a sheet having a thickness of 1 mm.

In an air-removed inactive atmosphere, the sheet was irradiated with electron beams at an irradiation dose of 20 kGy to 100 kGy by an electron accelerator (acceleration voltage of 2 MeV, and current value of 1 mA). The obtained crosslinked material by irradiating the sheet with the electron beams was used as the sheet of the example 1.

### EXAMPLES 2 THROUGH 5

The sample of each of the examples 2 through 5 was similar to that of the example 1 except that the concentration of the TAIC added to the polylactic acid was 1.5 wt %, 2 wt %, 3 wt %, and 5 wt % respectively.

### COMPARISON EXAMPLES 1 THROUGH 5

Except that the irradiation doses were 0 kGy to 10 kGy, the specimen of each of the comparison examples 1 through 5 was prepared in the same manner as that of the examples 1 through 5.

### COMPARISON EXAMPLE 6

Except that the irradiation dose was 0 to 100 kGy, the specimen of the comparison example 6 was prepared in the same manner as that of the example 1.

### COMPARISON EXAMPLES 7, 8

Except that the concentration of the TAIC was 0.5 wt % and 1 wt % respectively, the sample of each of the comparison examples 7, 8 was prepared in the same manner as that of the comparison example 6.

The manufacturing conditions of the examples and the comparison examples are shown in table 1.

TABLE 1

Irradiation doze of electron beam

| TAIC concentration | 0-10 kGy | 20-100 kGy |
|---|---|---|
| 0% | Comparison example 6 | |
| 0.5% | Comparison example 7 | |
| 1.0% | Comparison example 8 | |
| 1.2% | Comparison example 1 | Example 1 |
| 1.5% | Comparison example 2 | Example 2 |
| 2.0% | Comparison example 3 | Example 3 |
| 3.0% | Comparison example 4 | Example 4 |
| 5.0% | Comparison example 5 | Example 5 |

### (Evaluation of Examples and Comparison Examples)

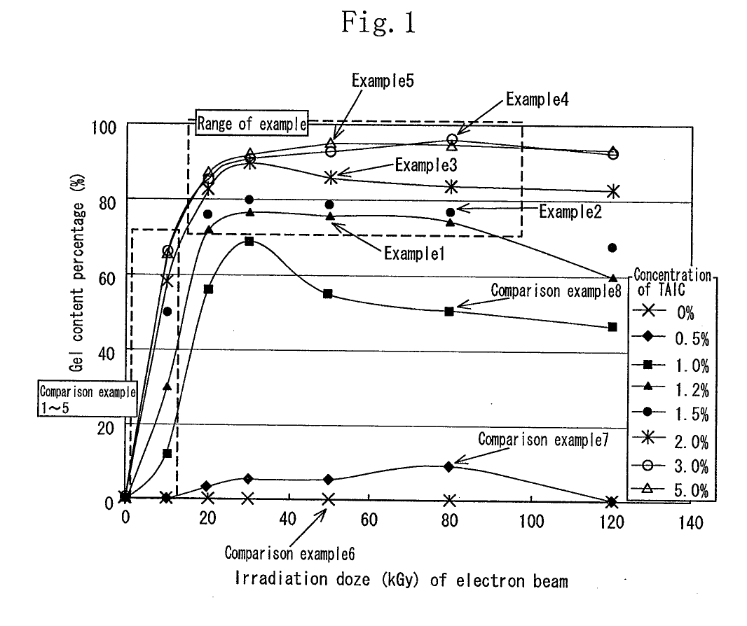

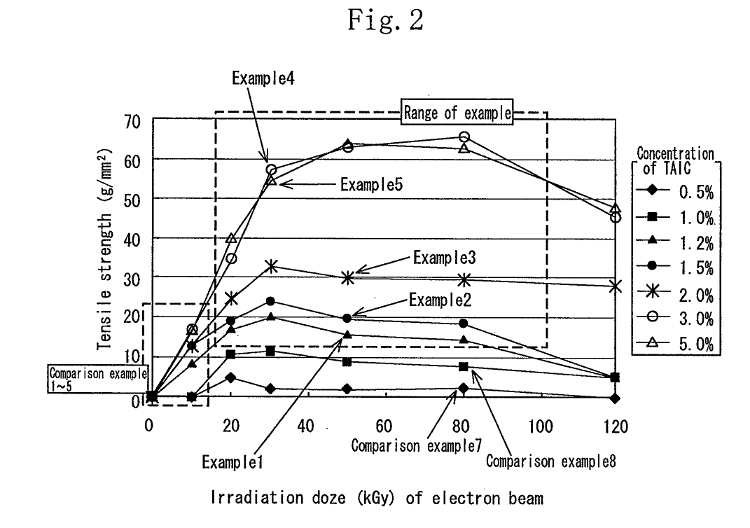

(1) gel fraction percentage and (2) tensile test at a high temperature were evaluated on each of the example and the comparison examples. The results are shown in FIGS. 1 and 2.

### (Evaluation of Tensile Test at High Temperature)

After samples were formed into a rectangle having a width of 1 cm and a length of 10 cm, the samples were pulled inside a constant-temperature tank of 180° C. at a tensile speed of 10 mm/minute, with chucks spaced at 2 cm to measure the break strength and break expansion thereof.

Measurement was made after the temperatures of the samples placed inside the constant-temperature tank reached 180° C.

Break strength (kg/cm²)=Tensile strength at broken time/(thickness of sample×width of sample)

Break expansion (%)=Distance between chucks at broken time−2 cm)/2 cm×100.

### (Results of Evaluation of Examples and Comparison Examples)

FIG. 1 shows the relationship among the irradiation dose of electron beams, the gel fraction percentage, and the concentration of the monomer in each of the examples and the comparison examples.

As shown in FIG. 1, in the sample of the comparison example 6 not containing the TAIC, a crosslinking reaction did not occur, and the gel fraction percentage was 0. In the sample of the comparison example 7 in which the concentration of the monomer was 0.5 wt %, a crosslinking reaction hardly occurred although the irradiation dose was large, and the gel fraction percentage was not more than 7%. In the sample of the comparison example 8 in which the concentration of the monomer was 1 wt %, the gel fraction percentage was not more than 70%.

In the samples of the comparison examples 1 through 5, the gel fraction percentage was 12 to 67% when the irradiation dose of radioactive rays was 10 kGy, although the concentration of the TAIC was not less than 1.2 wt %.

In the examples 1 through 5, irrespective of concentrations of the TAIC, the gel fraction percentage was maximum when the irradiation dose of the electron beams was in the range of 30 to 50 kGy, and the gel fraction percentages exceeded 75%. In the examples 5 and 6, the gel fraction percentage attained 95%. It was found that the effect obtained when the irradiation dose was 20 kGy was 80% to 90% of the effect obtained at the peak. In the examples 1, 2, and 3, with the increase of the irradiation dose, the gel fraction percentage decreased gradually. Although not shown in the graph, at 150 kGy, the gel fraction percentage was 50% to 60% of the gel fraction percentage at the peak, and at 200 kGy, the gel fraction percentage dropped to less than 50%, namely, about 30% of the gel fraction percentage at the peak.

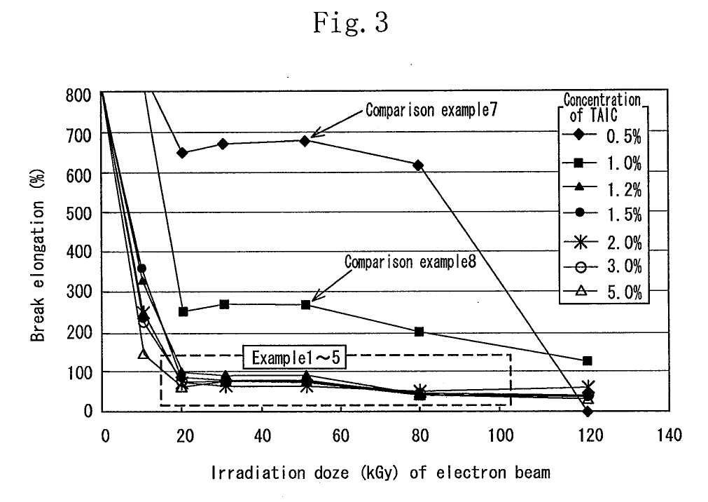

FIG. 2 shows the relationship among the tensile strength and the irradiation dose of electron beams at a high temperature in the examples and the comparison examples. FIG. 3 shows the relationship between the irradiation dose of the electron beams and the break expansion.

Of the comparison examples 1 through 6, in the sample not irradiated with electron beams, at 180° C. exceeding its melting point 160° C., the sample melted entirely, became soft, expanded, and was broken without generating a tensile strength. Although the break expansion was shown as infinity outside the graph for convenience, the break expansion could not be measured.

When the irradiation dose was 10 kGy, the sample of each of the examples 6 through 8 in which the concentration of the TAIC was less than 1.2 wt % was 0 in the strength thereof (tensile strength). On the other hand, the tensile strength of the sample of each of the comparison examples 1 through 5 in which the concentration of the TAIC was equal to that of sample of the examples fell in the range in which the tensile strength can be measured. But at this point, as shown in FIG. 3, the examples were expanded greatly. That is, when the samples deformed greatly, the tensile strength was generated. In the above-described range of the irradiation dose, the sheet deforms easily.

In the irradiation range in which the irradiation dose was not less than 20 kGy, namely, in the range of the examples 1 through 5, the expansion percentage lowers, and the tensile strength was generated. The tensile strength was in the range of 20 to 100 g/mm², and the expansion percentage was in the range of 30 to 100%.

Considering that an object of the present invention is to improve the deformability at a high temperature, it is important that the expansion percentage is small and that the tensile strength is large. Similarly to the gel fraction percentage, the tensile strength becomes large at 20 kGy. The peak was in the range of 30 to 50 kGy. The tensile strength lowered at not less than 100 kGy.

In the samples of the comparison examples 7 and 8, the break expansion shown in FIG. 3 does not become low unlike the samples of the examples 1 through 5. Thus the examples of the comparison examples 7 and 8 are insufficient in the heat resistance thereof. The sample of the comparison example 6 not containing the TAIC melted in any of the irradiation doses. Thus the tensile strength could not be measured and not shown in FIGS. 2 and 3.

From the tensile strength and the break expansion percentage at a high temperature in the samples of the examples and the comparison examples, it could be confirmed that the samples of the examples of the present invention were high in the configuration-retaining property (that is, high hardness) thereof, did not deform easily, and were heat-resistant.

The second embodiment will be described below.

The biodegradable material of the second embodiment is made of the heat-resistant crosslinked material of the second invention.

In the biodegradable material of the second embodiment, biodegradable aliphatic polyester and the hydrophobic polysaccharide derivative are integrated with each other by crosslinking to improve the configuration-retaining property (that is, high hardness) which deteriorates rapidly at temperatures not less than the glass transition point and impart heat resistance to the biodegradable material and further impart properties of not damaging transparency, surface glossiness, and smoothness thereto.

The biodegradable material of the second embodiment has a crosslinked structure having 50% to 95% in its gel fraction percentage (gel fraction dried weight/initial dried weight).

As described above, the gel fraction percentage of the polymer containing the biodegradable aliphatic polyester as its main component is set to not less than 50% and preferably 65%. Further the biodegradable aliphatic polyester and the hydrophobic polysaccharide derivative are integrated with each other by crosslinking so that innumerable three-dimensional screen structures are formed in the polymer. Therefore it is possible to impart the heat resistance which does not deform at temperatures not less than the glass transition temperature to the biodegradable material.

As the biodegradable aliphatic polyester, similarly to the first invention, the polylactic acid is preferably used.

As the hydrophobic polysaccharide derivative to be integrated with the biodegradable aliphatic polyester by crosslinking, etherified starch derivatives such as methyl starch, ethyl starch, and the like using starch such as corn starch, potato starch, sweet potato starch, wheat starch, rice starch, tapioka starch, sago starch as the material thereof; esterified starch derivatives such as acetate ester starch, aliphatic ester starch, and the like; and alkylated starch derivatives.

As the hydrophobic polysaccharide derivative, it is possible to utilize derivatives similar to starch whose material is cellulose and derivatives of other polysaccharides such as Pullulan.

The hydrophobic polysaccharide derivative can be utilized singly or by mixing two or more kinds thereof with each other. In view of the object of the present invention of mixing the hydrophobic polysaccharide derivative and the aliphatic polyester with each other, it is possible to preferably utilize derivatives, made sufficiently hydrophobic, in which hydroxyl groups are substituted at a substitution degree of not less than 1.5, favorably not less than 1.8, and more favorably not less than 2.0.

The substitution degree means an average value of the number of three hydroxyl groups, substituted by esterification, and the like which are contained in one constituting unit of the polysaccharide. Therefore the maximum value of the substitution degree is three. The derivative of the polysaccharide is affected by a functional group introduced thereinto by substitution. The derivative of the polysaccharide shows hydrophilic property when its substitution degree is not more than 1.5 and hydrophobic property when its substitution degree is not less than 1.5.

To improve the flexibility of the biodegradable material, similarly to the first invention, it is possible to add a plasticizer such as glycerin liquid at the normal temperature to the biodegradable resin or a plasticizer such as polyglycol and polyvinyl alcohol solid at the normal temperature thereto. In addition, it is possible to add a small amount of other biodegradable aliphatic polyesters such as polycaprolactam as a plasticizer to the polylactic acid. But this is not essential.

Similarly to the first invention, it is preferable to add the monomer having the allyl group to the aliphatic polyester and the hydrophobic saccharide. The monomer is capable of crosslinking the aliphatic polyester and the hydrophobic saccharide independently. Similarly to the first invention, particularly desirable monomers having the allyl group are the triallyl isocyanurate (hereinafter referred to as TAIC) and the trimethallyl isocyanurate (hereinafter referred to as TMAIC).

When not less than 0.1 wt % of the monomer is added to 100 wt % of the aliphatic polyester, the effect of the addition can be recognized. A more reliable effect can be obtained when the concentration of the monomer is in the range of 0.5 to 3 wt %. Considering the use of the biodegradable material as the biodegradable plastic, it is desirable to set the total of the biodegradable aliphatic polyester that is reliably decomposed and the hydrophobic polysaccharide derivative to not less than 99%. Thus the addition amount of the monomer is in the range of 0.5 to 1 wt %.

The biodegradable material consisting of the heat-resistant crosslinked material of the second embodiment is manufactured by uniformly mixing the biodegradable aliphatic polyester, the hydrophobic polysaccharide derivative, and the crosslinking-type polyfunctional monomer with one another at a temperature not less than the melting point of the biodegradable aliphatic polyester and then irradiating the mixture thereof with the ionizing radiation.

More specifically, initially, both the aliphatic polyester and the hydrophobic polysaccharide derivative are heated to a temperature at which the mixture thereof melts or softened by heating or dissolved and dispersed in a solvent dissolvable in chloroform, cresol or the like. Thereafter the monomer is added to the mixture, and the three components are mixed with one another as uniformly as possible. These three components may be mixed with one another together or two of the three components, for example, the aliphatic polyester and the hydrophobic polysaccharide derivative may be kneaded in advance to sufficiently mixedly disperse the hydrophobic polysaccharide derivative in the aliphatic polyester.

Thereafter the mixture is pressed and cooled rapidly to mold the mixture into a desired shape with the mixture softened by heating or dissolved in the solvent. Alternatively, the mixture is heated again to soften it, pressed, and then cooled rapidly to mold the mixture into a desired shape after the mixture is cooled or the solvent is removed by drying it. The molded material is irradiated with the ionizing radiation to generate the crosslinking reaction.

The ionizing radiation with which the molded material is irradiated is similar to that of the first invention. For example, γ-rays, x-rays, β-rays or α-rays can be used. In industrial production, the γ-rays emitted from cobalt 60 and electron beams emitted from an electron accelerator are preferable. The irradiation dose necessary for generating the crosslinking reaction is not less than 1 kGy nor more than 300 kGy, favorably not less than 30 kGy nor more than 100 kGy, and most favorably not less than 30 kGy nor more than 50 kGy.

Similarly to the first invention, instead of using the radioactive rays, the above-described chemical initiator may be used to generate the crosslinking reaction.

In the manufacturing method of the present invention, using the monomer such as the TAIC having the allyl group, the biodegradable aliphatic polyester and the hydrophobic polysaccharide derivative are integrally crosslinked with each other by irradiating the molded material with the ionizing radiation. Thereby the disadvantage of the aliphatic polyester of being poor in the configuration-retaining property (that is, high hardness) at temperatures not less than 60° C. is improved.

That is, the relationship among the biodegradable aliphatic polyester which is the main component of the biodegradable material, the hydrophobic polysaccharide derivative, the crosslinking-type polyfunctional monomer is as described below.

When the uniform mixture of the above-described three components is irradiated with the ionizing radiation, owing to the crosslinking-type polyfunctional monomer activated by the radioactive rays, crosslinked structures are formed among molecules of the biodegradable aliphatic polyester which is the main component of the biodegradable material, among molecules of the kneaded hydrophobic polysaccharide derivative, and among the molecules of the biodegradable aliphatic polyester and those of the hydrophobic polysaccharide derivative. Thereby innumerable three-dimensional network structures are formed.

By selecting the hydrophobic polysaccharide derivative which softens in the vicinity of the melting point of the biodegradable aliphatic polyester to be integrated therewith, both the hydrophobic polysaccharide derivative and the biodegradable aliphatic polyester can be kneaded by heating them. The hydrophobic polysaccharide derivative does not have a definite melting point and is very hard even at a high temperature. In the case of the biodegradable aliphatic polyester such as the polylactic acid which becomes soft at temperatures not less than the glass transition point 60° C. that is much lower than its melting point in the vicinity of 160° C. and loses its configuration-retaining property (that is, high hardness), the hydrophobic polysaccharide derivative imparts hardness to the entire uniform mixture because it has a softening point at not less than 160° C. and is hard and does not deform at a temperature below 160° C.

That is, in the present invention, the hydrophobic polysaccharide derivative is kneaded with the biodegradable aliphatic polyester. Further both hydrophobic polysaccharide derivative and the biodegradable aliphatic polyester are integrated with each other by the crosslinking-type polyfunctional monomer activated by the radioactive rays and captured into the network structure. Therefore it is possible to efficiently provide the entire polymer containing the biodegradable aliphatic polyester as its main component with the heat resistance which keeps the shape of the polymer hard at a temperature not less than the glass transition temperature and does not deform the polymer easily.

The method for mixing the hydrophobic polysaccharide derivative hard at high temperatures with the biodegradable aliphatic polyester is similar to the method of reinforcing the polylactic acid with the mineral filler disclosed in the above-described non-patent document. But the hydrophobic polysaccharide derivative is excellent in the following points:

(1) It is impossible to disperse the mineral filler in a size larger than its original size. On the other hand, the hydrophobic polysaccharide derivative melts when it is mixed with the aliphatic polyester by heating or by dissolving it in the solvent. Thus by selecting a mixing level as desired, the hydrophobic polysaccharide derivative can be mixed with the aliphatic polyester at a desired level in the size of particles and that of molecules before the hydrophobic polysaccharide derivative is mixed with the aliphatic polyester.

(2) There is no bonding between the mineral filler and the base consisting of resin, and the reinforcing effect depends mainly on the strength of the filler itself. On the other hand, crosslinking occurs between the hydrophobic polysaccharide derivative and the aliphatic polyester constituting the base of the biodegradable material by using the same monomer. Therefore owing to three effects including the hardness of the hydrophobic polysaccharide derivative, the improvement of its hardness provided by the crosslinking, and the integration of the hydrophobic polysaccharide derivative and the base consisting of the resin made by the crosslinking, the hydrophobic polysaccharide derivative is capable of providing the base consisting of the resin with a heat resistance exceeding the reinforcing effect obtained when the hydrophobic polysaccharide derivative serves as a filler.

(3) When the filler is mixed with the resin constituting the base of the biodegradable material and the mixture is molded, bleeding phenomenon that the filler oozes out of the resin occurs with time. On the other hand, for the same reason as that described in the above (2), in a mixing time, the hydrophobic polysaccharide derivative is not crosslinked and hence molecules thereof are dispersed. Thus the hydrophobic polysaccharide derivative can be readily mixed with the aliphatic polyester. But after it is irradiated with radioactive rays, molecules of the hydrophobic polysaccharide derivative are crosslinked to each other or integrally crosslinked with the aliphatic polyester. As a result, the hydrophobic polysaccharide derivative becomes polymeric. Thus the hydrophobic polysaccharide derivative never bleeds.

(4) The mixing of the mineral filler with the aliphatic polyester such as the polylactic acid causes the polylactic acid to lose its transparency and the glossiness of its surface, and thus the surface to feel rough. On the other hand, in the present invention, owing to a mixing condition, the biodegradable material loses its transparency only slightly and hence the appearance of the surface of the biodegradable material is not damaged.

(5) In the processability of the biodegradable material, the method of utilizing the mineral filler of nano size has succeeded in shortening the high-temperature maintaining time period in which the crystallinity is enhanced. The high-temperature maintaining time is not required in the present invention. Therefore the present invention is capable of greatly reducing the manufacturing time period.

The biodegradable material composed of the heat-resistant crosslinked material is capable of improving the configuration-retaining property (that is, high hardness) of the biodegradable aliphatic polyester, particularly the configuration-retaining property (that is, high hardness) of the polylactic acid at temperatures not less than 60° C. Further because the hydrophobic polysaccharide derivative is added to the polylactic acid to maintain the strength of the biodegradable material at a high temperature, the transparency of the polylactic acid and the glossiness of the surface thereof are not damaged greatly unlike the case in which the mineral filler is used for the polylactic acid. Furthermore although it is necessary to set a high temperature in an industrial production, the biodegradable material can be manufactured by using conventional injection molding equipment without deteriorating productivity.

Further because the hydrophobic polysaccharide derivative is also biodegradable, it hardly adversely affects an ecosystem in nature. Thus it is expected that the biodegradable material be used as a material substituting plastic products mass-produced and discarded. In addition, because the biodegradable material does not give a bad influence on the organism, it is suitably applied to medical appliances used inside and outside the organism.

In this embodiment, as the biodegradable aliphatic polyester, the polylactic acid is used, and the acetate ester starch is used as the hydrophobic polysaccharide derivative to integrate the polylactic acid and the acetate ester starch with each other. Further as the crosslinking-type polyfunctional monomer, the TAIC is used. 0.5 wt % to 3 wt % of the TAIC is used for 100 wt % of the polylactic acid.

The above-described three substances are mixed with one another. The mixture is injection molded to form a sheet. The sheet is irradiated with the ionizing radiation in an amount of 30 to 100 kGy. Crosslinking is accelerated by using the TAIC to integrate the polylactic acid and the acetate ester starch with each other by the crosslinking.

The obtained biodegradable material composed of the obtained heat-resistant crosslinked material has a gel fraction percentage of 50 to 95%, a substantial fusion molding temperature in the range of 150° C. to 200° C. which is not less than the melting point of the biodegradable aliphatic polyester and not less than the softening point of the hydrophobic polysaccharide derivative, a tensile strength of 30 to 70 g/mm² at a high temperature in the vicinity of 150° C. to 200° C., and an expansion percentage of 20 to 50%. That is, in environment having a high temperature, the biodegradable material is set low in its expansion percentage, high in its tensile strength, and high in its configuration-retaining property (that is, high hardness).

Examples (6 through 11) of the second embodiment and comparison examples (9 through 18) were prepared.

### EXAMPLE 6

As the aliphatic polyester, finely powdered polylactic acid (Racia H-100J manufactured by Mitsui Kagaku) was used. As the hydrophobic polysaccharide derivative, powder of acetate ester starch (CP-1 produced by Nippon Corn Starch Inc.) was used.

In the polysaccharide derivative, the substitution degree of the hydroxyl group is about 2.0. The derivative of the polysaccharide is not soluble in water, but dissolves in acetone. Thus the polysaccharide derivative is hydrophobic. The polysaccharide derivative softens at temperatures not less than 180° C., does not have a definite melting point, and has a very high Young's modulus.

Five wt % of the acetate ester starch was mixed with 100 wt % of the polylactic acid. The mixture was melted at 190° C. by using a Lab Plast mill that is a closed kneader, and sufficiently kneaded until it became transparent. Thereafter 3 wt % of the TAIC (manufactured by Nippon Kasei Inc.) which is the monomer having the allyl group was added to the mixture of the polylactic acid and the acetate ester starch. The components were kneaded for 10 minutes at 20 rpm sufficiently and mixed with one another.

Thereafter the uniform mixture was thermally pressed at 190° C., and thereafter cooled rapidly at about 100° C./minute to the normal temperature. Thereby a sheet having a thickness of 1 mm was obtained. In an air-removed inactive atmosphere, the sheet was irradiated with electron beams in an amount of 50 kGy by using an electron accelerator (acceleration voltage of 2 MeV, and current value of 1 mA). The obtained crosslinked material was used as the sample of the example 6.

### EXAMPLES 7 AND 8

The sample of each of the examples 7 and 8 was prepared in a manner similar to that of the example 6 except that the ratio of the hydrophobic polysaccharide derivative to the aliphatic polyester was set to 10 wt % in the example 7 and 30 wt % in the example 8.

### EXAMPLE 9

The sample of the example 9 was prepared in a manner similar to that of the example 6 except that as the hydrophobic polysaccharide derivative, cellulose diacetate (acetate cellulose L-30 produced by Dicel Inc.) having a substitution degree of about 2 was used and that the ratio of the hydrophobic polysaccharide derivative to the aliphatic polyester was set to 10 wt %.

### EXAMPLE 10

In the example 10, as the hydrophobic polysaccharide derivative, the cellulose diacetate having a substitution degree of about 2 equal to that of the sample of the example 9 was used. The ratio of the hydrophobic polysaccharide derivative to the aliphatic polyester was 30 wt %. Except this, the sample was prepared in a manner similar to that of the example 6.

### EXAMPLE 11

Polybutylene succinate (Bionore #1020 produced by Showa Kobunshi Inc.) was used as the aliphatic polyester. The aliphatic ester starch (CP-5 produced by Nippon Corn Starch Inc) was used as the hydrophobic polysaccharide derivative. The aliphatic ester starch had a substitution degree of 2 and an average length of hydrocarbon at about 10.

In a manner similar to that of the example 6, the weight ratio of the TAIC to the total of the aliphatic polyester and the hydrophobic polysaccharide derivative was 3. Except this, the sample was prepared.

The aliphatic polyester and the hydrophobic polysaccharide derivative were kneaded at 150° C. that is the softening point and the mixture was pressed at 150° C. In this manner, the sheet was obtained.

### COMPARISON EXAMPLES 9 THROUGH 14

Except that the sample was not irradiated with electron beams, the sample of each of comparison examples 9 through 14 was prepared in a manner similar to that of the example 6 through 11.

### COMPARISON EXAMPLE 15

The sample of the comparison example 15 was prepared in a manner similar to that of the example 6 except that the hydrophobic polysaccharide derivative and the monomer were not used and that only the polylactic acid was used as the material for the sample.

### COMPARISON EXAMPLE 16

The hydrophobic polysaccharide derivative was not used for the sample of the comparison example 16.

### COMPARISON EXAMPLE 17

The sample of the comparison example 17 was prepared in a manner similar to that of the example 8 except that 3 wt % of the TMPT was used instead of the TAIC.

### COMPARISON EXAMPLE 18

The sample of the comparison example 18 was prepared in a manner similar to that of the example 11 except that the crosslinking-type polyfunctional monomer was not used.

The difference between the examples 6 through 11 and the comparison examples 9 through 18 was shown in table 2.

TABLE 2

| | | | | | | Evaluation of | |
|---|---|---|---|---|---|---|---|
| | | | | | | configuration- | |
| | | Hydrophobic | | | | retaining property | |
| | | polysaccharide derivative | | | | (that is, high | |
| | Aliphatic | | Mixing | Monomer and | Irradiation dose | hardness) | |
| | polyester | Kind | amount | concentration | of electron beam | 80° C. | 150° C. |
| Example | | | | | | | |
| 6 | Polylactic | Acetate ester | 5 wt % | TAIC 3% | 50 kGy | ◯ | Δ |
| 7 | acid | starch | 10 wt % | | | ◯ | ◯ |
| 8 | | | 30 wt % | | | ◯ | ◯ |
| 9 | | Acetate ester | 10 wt % | | | ◯ | ◯ |
| 10 | | cellulose | 30 wt % | | | ◯ | ◯ |
| 11 | Polybutylene | Fatty acid ester | 30 wt % | | | ◯ | ◯ |
| | succinate | starch | | | | | |
| Comparison | | | | | | | |
| Example | | | | | | | |
| 9 | Polylactic | Acetate ester | 5 wt % | TAIC 3% | 0 kGy | X | X |
| 10 | acid | starch | 10 wt % | | | X | X |
| 11 | | | 30 wt % | | | X | X |
| 12 | | Acetate ester | 10 wt % | | | X | X |
| 13 | | cellulose | 30 wt % | | | X | X |
| 14 | Polybutylene | Fatty acid ester | 30 wt % | | | X | X |
| | succinate | starch | | | | | |
| 15 | Polylactic | Not used | | Not used | 50 kGy | X | X |
| 16 | acid | | | TAIC 3% | | ◯ | X |
| 17 | | Acetate ester | 30 wt % | TMPT 3% | | ◯ | X |
| | | starch | | | | | |
| 18 | Polybutylene | Fatty acid ester | 30 wt % | Not used | | ◯ | X |
| | succinate | starch | | | | | |

In table 2, reference symbol ◯ denotes that the shape of the sample did not change before and after the test. Reference symbol Δ that the shape of the sample changed to some extent. For example, it was bent or the like. Reference symbol X denotes that the sample fell down completely and did not maintain its original shape.

To evaluate the effect of improving the heat resistance of the samples of the examples 6 through 11 and those of the comparison examples 9 through 18 at temperatures not less than the glass transition point, the configuration-retaining property (that is, high hardness) of each sample at 80° C. and 150° C. was evaluated.

The evaluation was made on the samples of the examples and the comparison examples that were irradiated with electron beams having an irradiation dose of 0 kGy and 50 kGy. Table 2 shows the results.

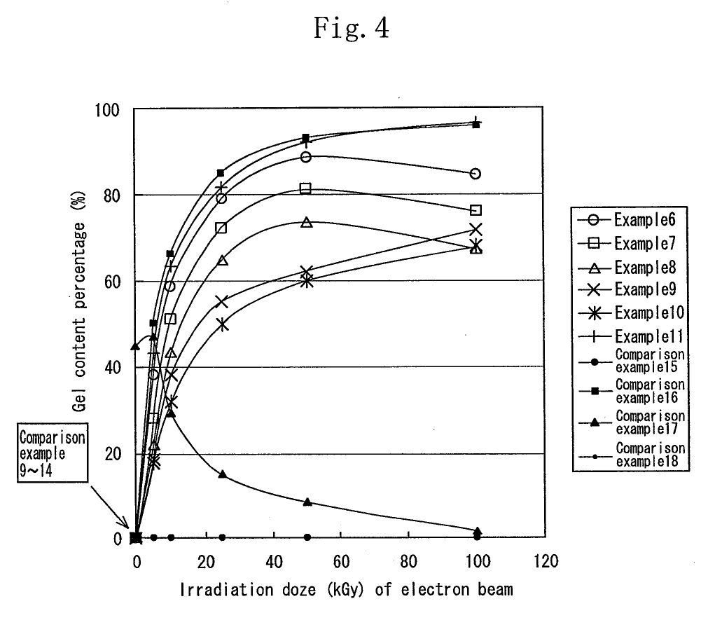

To evaluate the crosslinking degree of molecules irradiated with the electron beams, the relationship between the amount of irradiation applied to the examples and the gel fraction percentage was measured in each of the examples and the comparison examples. FIG. 4 shows the results.

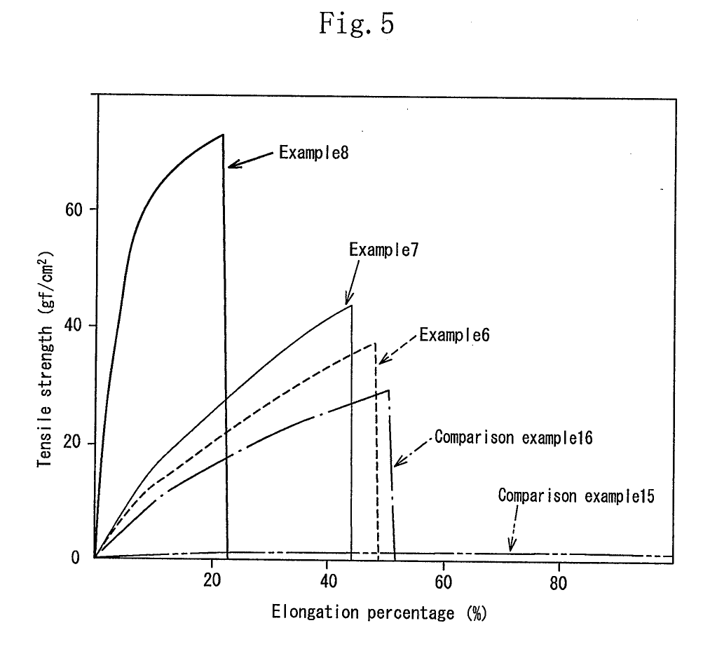

To examine the effect of improving Young's modulus at temperatures not less than the glass transition point, a strength expansion curve in a tensile test was measured at 100° C. on the samples of the examples 6 through 8 and the comparison examples 15 and 16 to which electron beams were applied in an amount of 50 kGy. FIG. 5 shows the results.

The evaluation method is as described below.

(Evaluation of Configuration-Retaining Property (that is, High Hardness))

The sample sheet of each of the examples and the comparison examples cut in the shape of a rectangle having a length of 10 cm and a width of 1 cm was erected almost vertically in a groove having a width of 1 mm equal to the thickness of the sheet and a depth of 1 cm, with the longer side of the sheet vertical. The sheet and the groove were put into a constant temperature bath having a temperature 80° C. to check whether the sheet was erect for itself. The evaluation was made at 80° C. and 150° C.

The evaluation of the gel fraction percentage and the evaluation of the tensile test at high temperature are as described above.

### (Evaluated Results of Examples and Comparison Examples)

Regarding the configuration-retaining property (that is, high hardness), as shown in table 2, at 80° C. higher than 60° C. which is the glass transition point of the polylactic acid, the samples of all of the examples 6 through 11 and the comparison examples 16 through 18 did not have any change a little before and a little after the time when heating started, whereas the samples of the comparison examples 9 through 15 melted and fell down and did not retain the original shape. At 150° C. in the neighborhood of the melting point, the sheet of the example 6 was bent and had a change in its shape, whereas the sheets of the examples 7 through 11 showed preferable configuration-retaining property (that is, high hardness).

Regarding the gel fraction percentage, as shown in FIG. 4, the crosslinking of the sample of the examples 6 through 11 progressed by the irradiation of the electron beam, with the result that the aliphatic polyester, the hydrophobic polysaccharide derivative, and the crosslinking-type polyfunctional monomer were mixed and integrated with one another. The peak attained 68 to 95%. In the examples 6 through 8, the gel fraction percentage attained the peak when the irradiation dose was in the vicinity of 50 kGy. In the examples 9 through 11, the gel fraction percentage attained the peak when the irradiation dose was 100 kGy. When the irradiation dose exceeded 100 kGy, the samples containing the polylactic acid which is decomposed by radioactive rays started to decompose and the gel fraction percentage lowered.

The sample of the comparison example 16 containing the polylactic acid and the TAIC crosslinked similarly to the examples. In the sample of the comparison example 9, crosslinking occurred in the TMPT owing to heat generated in the process of manufacturing the sample. Thus when the sample was irradiated with electron beams, the sample lost a crosslinking function and was decomposed by irradiation.

Regarding the tensile strength and the expansion, as shown in FIG. 5, at a measuring condition of 100° C., the sample of the comparison example 15 consisting of the polylactic acid had little tensile strength and expanded greatly when it was pulled. The sample of the comparison example 16 which contained the polylactic acid and the TAIC and crosslinked showed a tensile strength to some extent, but the extent of the tensile strength was insufficient.

On the other hand, in the examples 6 through 8, the tensile strength was 30 to 70 g/mm², and the expansion percentage was 20 to 50%. As the addition amount of the hydrophobic polysaccharide derivative became larger, the tensile strength became increasingly high, and the degree of the expansion dropped. That is, it was admitted that Young's modulus increased and the configuration-retaining property (that is, high hardness) increased.

From the evaluation of the sheets of the examples and the comparison examples, the polylactic acid decreases sharply in its Young's modulus at not less than 60° C. and becomes very soft. Thus the polylactic acid has difficulty in maintaining its original shape. It could be confirmed that although the configuration-retaining property (that is, high hardness) increased to some extent owing to crosslinking caused by the addition of the monomer such as the TAIC thereto, the degree of the configuration-retaining property (that is, high hardness) was insufficient.

The acetate ester starch and the acetate ester cellulose which are the hydrophobic polysaccharide derivatives are crosslinked by means of the TAIC and show a very high Young's modulus at not less than the glass transition point of the polylactic acid. It could be confirmed that in the neighborhood of the melting point of the polylactic acid, the acetate ester starch and acetate ester cellulose do not show a clear melting point and Young's modulus thereof do not lower much.

The third embodiment will be described below.

The biodegradable material of the third embodiment is a heat-resistant material of the third invention which is used as a heat-shrinkable material having a high heat shrink characteristic. The biodegradable material of the third embodiment is composed of a mixture of the biodegradable aliphatic polyester and the low-concentration monomer having the allyl group. The mixture is irradiated with ionizing radiation or a chemical initiator is added to the mixture to allow the mixture to have a crosslinked structure. Thereafter the mixture is expanded by heating it. When the mixture is heated at a temperature not less than the temperature used at an expanding time, the mixture shrinks in a range of not less than 40% nor more than 80%.

More specifically, as the biodegradable aliphatic polyester, the polylactic acid is used. The gel fraction percentage (gel fraction dried weight/initial dried weight) obtained by crosslinking is 10 to 90%. The shrinkage factor at a temperature not more than 140° C. is less than 10%. The shrinkage factor at a temperature not less than 160° C. is 40 to 80%.

As the aliphatic polyester used as the biodegradable polymer, the above-described polylactic acid is used similarly to the first and second embodiments. To improve the flexibility of the biodegradable aliphatic polyester, a plasticizer similar to that of the first and second embodiments may be added thereto.

As the crosslinking-type polyfunctional monomer to be mixed with the aliphatic polyester, the monomer having the allyl group similar to that of the first and second embodiments is used.

When the concentration ratio of the monomer having the allyl group is 0.5 wt % for 100 wt % of the polylactic acid, a crosslinking reaction hardly occurs. Thus to set the gel fraction percentage to 10 to 90% to achieve the object of the present invention of obtaining a high heat resistance and a high shrink characteristic, 0.5 wt % is insufficient as the concentration of the monomer. It is preferable to set the concentration of the monomer to 0.7 wt % to 3 wt %.

Even if the concentration of the monomer is increased to not less than 3 wt %, there is not an outstanding difference in the effect. When the addition amount of the monomer is as high as about 5 wt %, the gel fraction percentage increases immediately to not less than 80% and cannot be controlled easily.

To increase the shrinkage factor, the gel fraction percentage is preferably 50 to 70%. To this end, the addition amount of the monomer is favorably in the range of 0.7 to 2 wt % and most favorably 0.8 to 0.9 wt %.

The degree of the crosslinking can be evaluated based on the above-described gel fraction percentage.

Although the mixture is irradiated with the ionizing radiation to allow it to have the crosslinked structure, a chemical initiator similar to that of the first and second inventions may be mixed with the aliphatic polyester to generate the crosslinking reaction.

When the ionizing radiation is used, similarly to the first and second inventions, as the ionizing radiation to be used for the crosslinking, γ-rays, x-rays, β-rays or α-rays can be used. In industrial production, the γ-rays emitted from cobalt 60 and electron beams emitted from an electron accelerator are preferable. The irradiation dose of the ionizing radiation depends on the concentration of the monomer to some extent. The crosslinking occurs at even 1 to 150 kGy. But the effect of the crosslinking and the effect of improving the strength of the biodegradable material at a high temperature can be obtained at not less than 5 kGy and more desirably at not less than 10 kGy at which the effects can be securely obtained.

The polylactic acid preferable as the aliphatic polyester will collapse by itself with radioactive rays. Thus when the irradiation dose is more than the necessary irradiation dose, the decomposition of the polylactic acid will progress in reverse to the crosslinking. Accordingly the upper limit of the irradiation dose is 80 kGy and favorably 50 kGy.

Therefore the irradiation dose of electron beams is set to not less than 5 kGy nor more than 50 kGy, favorably not less than 10 kGy nor more than 50 kGy, and most favorably not less than 15 kGy nor more than 30 kGy.

The polylactic acid is of a type which is decayed by radioactive rays. But even though a part thereof is decomposed, an apparent gel fraction percentage does not lower when the polylactic acid is partly connected with a crosslinked network. In view of the object of the present invention of storing the shape of the biodegradable material, rather than the structure which has a portion partly connected with the network and has many gelled portions unuseful for storing the shape, a structure in which crosslinked molecules of the polylactic acid are connected with one another at many points to form a strong reticulate framework and which has many non-crosslinked portion that is freely movable at a heating time is preferable in that the structure has a high shrink force and a large deformation amount and thus has a high shrinkage factor. Therefore in the present invention, the structure has an ideal state at the time immediately after the crosslinking reaction of the monomer finishes.

More specifically, in the graph shown in FIG. 1 (FIG. 4) in which the abscissa is the irradiation dose and the ordinate is the gel fraction percentage, as the irradiation dose becomes larger, the gel fraction percentage becomes increasingly high and saturates and does not increase. It can be the ideal state is obtained at a point immediately before the gel fraction percentage remains unchanged, namely, in the vicinity of the inflection point of the graph.

The ideal state of the gel fraction percentage differs in dependence on the concentration of the monomer. At a high concentration, the gel fraction percentage saturates at a high gel fraction percentage. At a low concentration, the gel fraction percentage saturates at a low gel fraction percentage.

According to the investigation of the present inventors, as described above, the ideal gel fraction percentage is in the range of 50 to 70%. The ideal state, namely, the inflection point in the graph is obtained when the concentration of the monomer is in the range of 0.7 to 1.3 wt %, as described above.

When the irradiation of the ionizing radiation is continued after the crosslinking reaction finishes, the molecules of the polylactic acid are decomposed. Even though it is considered that crosslinking has occurred in terms of the gel fraction percentage and the gel fraction percentage becomes high, the crosslinked network is broken at many points. Thus crosslinked molecules do not contribute to storage of the shape. Therefore when a gel fraction percentage which has been 50 to 70% becomes 50 to 70% again as a result of an excessive reduction after passing a peak owing to an increase of the irradiation dose, the gel fraction percentage is inappropriate.

As described above, by setting the gel fraction percentage to the range of 10 to 90% and preferably 50 to 70%, innumerable three-dimensional networks are generated in the polymer. Thereby the polymer can be provided with heat resistance to such an extent that the polymer does not deform at temperatures not less than the glass transition temperature.

As will be described later, because at an expanding time, the polylactic acid is heated at temperatures not less than the melting point thereof to expand it, the crystalline portion of the polylactic acid as well as the non-crystalline portion thereof melt and thus the polylactic acid is expanded. When the mixture is cooled in the expanded state, the non-crystalline portion of the polylactic acid and the crystalline portion thereof become hard, and the expanded state is maintained. The three-dimensional structure made firm by the monomer stores an expansion-caused strain. When the mixture is heated again, the non-crystalline portion of the polylactic acid melts, but the expansion is maintained by the crystalline portion thereof. When the crystalline portion melts at the melting point, the strain stored in the three-dimensional network structure is released and the polylactic acid shrinks and returns to its original shape.

For example, when the temperature at an expanding time is set to 160 to 180° C., the biodegradable heat-shrinkable material containing the polylactic acids shrinks at not less than 160° C. Owing to the strong three-dimensional network structure, the shrinkage factor can be greatly increased to 40 to 80%.

In the method of the third invention for manufacturing the heat-shrinkable biodegradable material, a crosslinking-type polyfunctional monomer is added at a low concentration to a biodegradable material and the crosslinking-type polyfunctional monomer and the biodegradable material are kneaded; and the mixture is molded into a predetermined shape by heating the mixture under pressure and the mixture rapidly is cooled; the mixture is irradiated with ionizing radiation to generate a crosslinking reaction so that a gel fraction percentage is set to not less than 10% nor more than 90%; the mixture is expanded while the mixture is being heated at a temperature not less than a fusing temperature of the biodegradable material nor more than a temperature equal to an addition of the fusing temperature and 20° C., after the mixture is irradiated with the ionizing radiation to form the mixture as a heat-shrinkable material.

According to the manufacturing method, when the uniform mixture is heated at a temperature not less than the temperature used in the expanding time, the uniform mixture can be formed as the heat-shrinkable material which shrinks in the range of not less than 40% nor more than 80%.

In the method for manufacturing the biodegradable heat-shrinkable material having a heat shrinkage factor at 40 to 80%, the monomer having the allyl group is added to the biodegradable aliphatic polyester at a low concentration, and the monomer having the allyl group and the biodegradable aliphatic polyester are kneaded and the mixture is molded into a predetermined shape;

the mixture is irradiated with ionizing radiation at not less than 1 kGy nor more than 150 kGy to generate a crosslinking reaction so that a gel fraction percentage is set to not less than 10% nor more than 90%; and after the mixture is irradiated with the ionizing radiation, the mixture is expanded while the mixture is being heated at a temperature in the range of 60° C. to 200° C. to form the mixture as a heat-shrinkable material.

The heat-shrinkable material shrinks at a shrinkage factor in the range of 40% to 80%, when the heat-shrinkable material is heated at a temperature not less than a temperature used at an expanding time.

When the polylactic acid is used as the biodegradable aliphatic polyester, not less than 0.7 nor more than 3.0 wt % of the monomer having the allyl group is added to 100 wt % of the polylactic acid, and the polylactic acid and the monomer having the allyl group are kneaded;

the mixture is molded into a thin film, a thick sheet or a tube, and thereafter the thin film, the thick sheet or the tube is irradiated with ionizing radiation at not less than 5 kGy nor more than 50 kGy to generate a crosslinking reaction so that a gel fraction percentage thereof is set to not less than 50% nor more than 70%; and

after the crosslinked structure is obtained, the thin film, the thick sheet or the tube is heated at not less than 150° C. nor more than 180° C. to expand the thin film, the thick sheet or the tube at an expanding magnification of two to five.

It is more favorable to use triallyl isocyanurate as the monomer having the allyl group, set the addition amount of the triallyl isocyanurate to not less than 0.7 wt % nor more than 2.0 wt % for 100 wt % of the polylactic acid, irradiating the mixture with electron beams at not less than 10 kGy nor more than 30 kGy, and heat the mixture at not less than 160° C. nor more than 180° C. at the expanding time.

The reason the gel fraction percentage at the time when the crosslinking reaction finishes is set to the range of 10 to 90% and preferably 50 to 70% is because as described above, in this range, it is possible to improve the crosslinking, enhance the heat resistance, and increase the heat shrink characteristic. By setting the gel fraction percentage to the vicinity of 60%, it is possible to obtain heat shrink characteristic at 40 to 80% by heating the mixture at a temperature not less than 160° C.

In the evaluation of the expansion characteristic, the gel fraction percentage at 50 to 70% is marked as ⊚, the gel fraction percentage at 10 to 50% and 70 to 90% is marked as ◯, the gel fraction percentage at 6 to 10% is marked at Δ, and the gel fraction percentage at 0 to 5% and 90 to 96% is marked as X.

Because the shape is stored by the network obtained by the crosslinking, the degree of the crosslinking is lower than 50%. When the degree of the crosslinking is set less than 10%, the shrink characteristic and the heat resistance are lost. On the other hand, when the degree of the crosslinking is set more than 70%, and specifically more than 90%, the crosslinking progresses excessively and hence the shape becomes firm and hardly deforms. Consequently the expansion characteristic and the shrink characteristic deteriorate. Thus it was admitted that the range in which the heat resistance and the heat shrink characteristic can be imparted is 10 to 90% and that in the range of 50 to 70%, the polymer has excellent heat resistance and heat shrink characteristic.

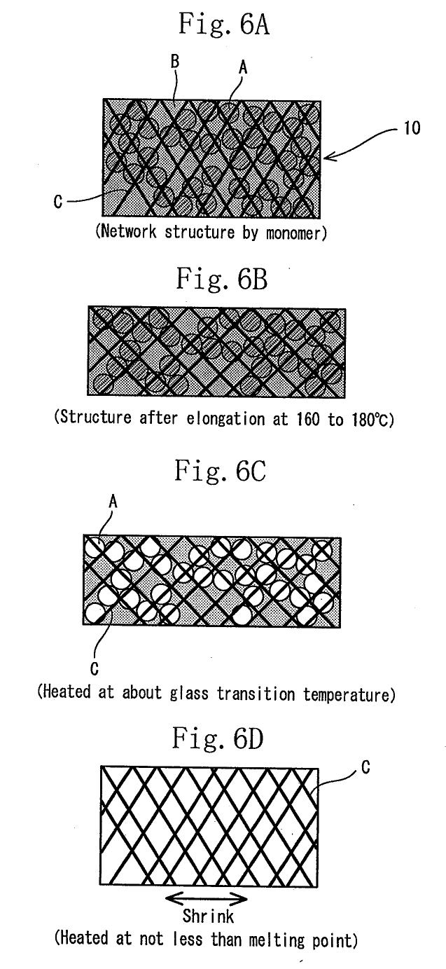

FIG. 6 shows the relationship among the network structure, the expansion, and the thermal shrinkage according to the gel fraction percentage before the expansion. In FIG. 6, black dots denote a crystalline portion A, the portion other than the black dots denotes a non-crystalline portion B, and oblique lines denote a network C. When a sheet 10 shown in FIG. 6(A) having a crosslinked structure having a gel fraction percentage of 50 to 70% is expanded by heating it at 160° C. to 180° C., as shown in FIG. 6B, the inclination of the network C changes and has an expanded state. When the expanded sheet is heated at a temperature not less than 60° C. which is the glass transition temperature of the polylactic acid, as shown in FIG. 6(C), the non-crystalline portion B melts. When the expanded sheet is heated at a temperature not less than 160° C. that is the fusing temperature of the polylactic acid, the crystalline portion A melts. But molecules of the network C are completely bonded to each other, the network C does not melt. Because the configuration-storing property of the network C is high, the expanded network C returns to the original shape shown in FIG. 6(D) and shrinks.

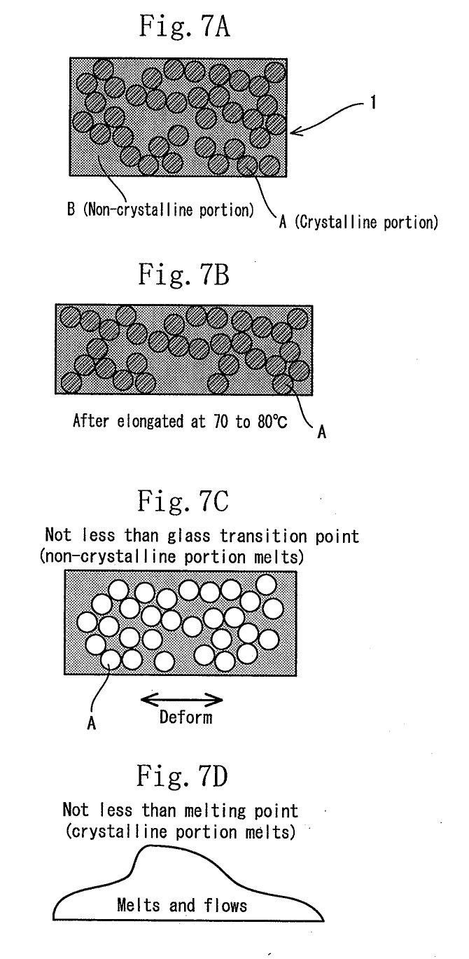

FIG. 7 shows a sheet which is made of the polylactic acid and not crosslinked. After the sheet shown in FIG. 7(A) is expanded in a heating condition of 70° C. to 80° C. as shown in FIG. 7(B), the non-crystalline portion B melts in the neighborhood of the glass transition temperature of the polylactic acid and deforms its shape, as shown in FIG. 7(C). As shown in FIG. 7(D), when the sheet is heated at a temperature not less than the melting point, the crystalline portion A melts.

The reason the heating condition at the expanding time after the crosslinking finishes is set to 60° C. to 200° C., favorably not less than 150° C. nor more than 180° C., and most favorably not less than 160° C. nor more than 180° C. is attributed to the fact that the temperature (glass transition temperature) at which the non-crystalline portion of the crosslinked polylactic acid starts to transfer is a little less than 60° C. and the melting point at which the crystal melts is 150 to 160° C.

When the sheet is expanded in the range (60 to 150° C.) from the glass transition temperature to the melting point, the non-crystalline portion thereof melts at the glass transition temperature and deforms. Thus the sheet starts to thermally shrink at 60° C. But the crystalline portion thereof does not shrink and hence the heat shrinkage factor does not increase. Therefore to increase the heat shrinkage factor, the sheet is expanded at not less than 150° C. at which the crystalline portion thereof melts and thereafter the sheet is shrunk at 150 to 160° C. Thereby the heat shrinkage factor can be increased to 40 to 80%.

Therefore the heating temperature at the expanding time is set to favorably not less than 150° C. If the heating temperature at the expanding time is set to 200° C., it is necessary to expand the sheet in a short period of time. Thus the heating temperature at the expanding time is set to not more than 180° C. and most favorably not less than 160° C. nor more than 180° C. not less than the melting point.

When the mixture is expanded at the above-described heating temperature, the expanding magnification is set to two to five. This corresponds to the fact that the heat shrinkage factor of the polylactic acids composing the biodegradable heat-shrinkable material is set to 40 to 80%.

The heat shrinkage factor is not more than 5% at temperatures not more than 140° C. irrespective expansion percentages. At 150° C., the shrinkage factor is about 40%. But when the sheet is heated to not less than 160° C., the shrinkage factor is 65 to 70%. Thus the expanding magnification is set to not less than two nor more than three, and favorably not more than 2.5.

The sheet is expanded by using any of uniaxial expansion, biaxial expansion, and multi-axial expansion and can be expanded a roll method, a Denter method or a tube method.

As described above, in the biodegradable material of the third embodiment, when the mixture of the above-described components is irradiated with the ionizing radiation, owing to the mixing of the monomer having the allyl group, the crosslinking of the biodegradable aliphatic polyester such as the polylactic acid is accelerated and hence the gel fraction percentage can be set to 10 to 90%. Thus the length of the mixture can be expanded to about five times as large as its original-length. Further when the expanded heat-shrinkable material is heated to a temperature not less than the melting point, it can be thermally shrunk at a shrinkage factor of 40 to 80% owing to the network storing the shape. Further a change of the shape is prevented by the crystalline portion and the network that do not fuse at the glass transition point of the glass transition temperature of the polylactic acid. Thereby the biodegradable material is resistant to heat.

In this embodiment, the TAIC (triallyl isocyanurate) is added to the polylactic acid at a low concentration. 0.7 to 0.9 wt % of the TAIC is added to 100 wt % of the polylactic acid.

After the TAIC is added to the dissolved polylactic acid, they are kneaded. Thereafter the mixture is molded (thermally pressed) by heating the mixture at 180° C. under pressure and thereafter cooled rapidly at about 100° C./minute to the normal temperature to obtain a sheet having a required thickness.

In an air-removed inactive atmosphere, the sheet is irradiated with electron beams at an irradiation dose of 10 to 30 kGy, an applied voltage of 2 MeV, and a current value of 1 mA to progress the crosslinking of molecules of the polylactic acid by means of the TAIC. When the crosslinking finishes, the gel fraction percentage is 50% to 70%.

The sheet irradiated with the electron beams is heated at 160° C. to 180° C. to expand the sheet at an expanding magnification of not more than five by uniaxial expansion. After the sheet is expanded, the sheet is cooled to the room temperature to obtain the biodegradable heat-shrinkable material.

The present invention is not limited to the above-described embodiment. By changing the kind of the material for the biodegradable material and the kind of the monomer having the allyl group, it is possible to alter the irradiation dose of electron beams, the gel fraction percentage which is obtained owing to the crosslinking caused by the irradiation of the electron beams, the heating temperature at the expanding time, and the expanding magnification within the scope of the present invention. At that time, the heating temperature at the expanding time is set to not less than the melting point of the material for the biodegradable material and to the neighborhood of the melting point. The mixture of the components is expanded under this heating condition. In this manner, the heat-shrinkable material is manufactured. Thereby when the heat-shrinkable material is heated at not less than the above-described temperature, the heat shrinkage factor thereof can be increased to about 80%.

### (Examples and Comparison Examples)

42 kinds of samples of the examples and the comparison examples of the third embodiments were prepared as shown in table 3 shown below.

As the aliphatic polyester, finely powdered polylactic acid (Racia H-100J manufactured by Mitsui Kagaku) was used. The TAIC (manufactured by Nippon Kasei Inc.) which is the monomer having the allyl group was added at 0 wt %, 0.5 wt %, 1.0 wt %, 2.0 wt %, and 3.0 wt % to the polylactic acid which was melted at 180° C. by using a Lab Plast mill which is a closed kneader and sufficiently kneaded until it became transparent. The mixture was sufficiently kneaded at 20 rpm for 10 minutes.

Thereafter the uniform mixture was thermally pressed at 180° C. to obtain a sheet having a thickness of 1 mm. In an air-removed inactive atmosphere, the sheet was irradiated with electron beams by using an electron accelerator (acceleration voltage: 2 MeV, and current value: 1 mA). The irradiation dose was set to 0 kGy, 10 kGy, 20 kGy, 30 kGy, 50 kGy, 80 kGy, and 120 kGy, as shown in table 3.

Thereafter the sheets irradiated with the electron beams were heated at 180° C. to expand the sheet up to 2.5 times as long as the original length thereof. After the sheets were expanded, the temperature was dropped to the room temperature with the sheets fixed in that state. In this manner, the heat-shrinkable samples were manufactured.

TABLE 3

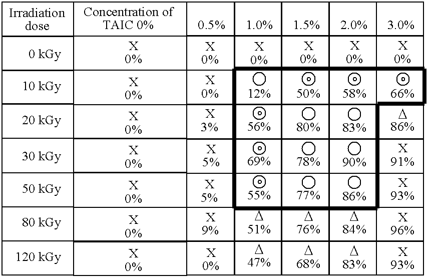

The expansion characteristic of the 42 kinds of the samples was evaluated, and the gel fraction percentage thereof was measured. Table 3 shows the results. The gel fraction percentage was measured by the above-described method. The gel fraction percentage was shown in the lower line of each sample.

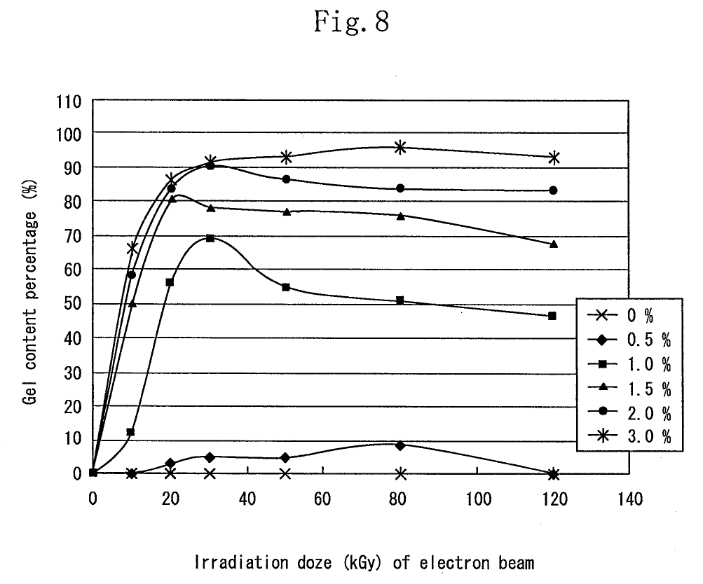

The relationship between the gel fraction percentage and the irradiation dose of electron beams is shown in the graph of FIG. 8.

### [Method of Evaluating Expansion Characteristic]

Regarding samples which could not be expanded to 2.5 times as long as the original length, magnifications at which they could be expanded without the samples being broken were evaluated by stages. The magnifications are shown at the upper line of each sample.

X=sample which could be hardly expanded

Δ=sample which was broken at an expanding magnification of 1.2 to 2.0 times as long as the original length

◯=2.0 to 2.5 times as long as the original length

⊚=not less than 2.5 times as long as the original length

In table 3, samples of the examples are surrounded with a double line and evaluated as ⊚ and ◯, Samples of the comparison examples are disposed on the periphery of the double line and marked as Δ and X.

In some samples of the comparison examples marked as Δ and X, the irradiation dose of the electron beams was 0 kGy or the addition amount of the TAIC was not more than 0.5 wt %. In some samples of the comparison examples, the irradiation dose of electron beams was 80 kGy and 120 kGy irrespective of the addition amounts of the TAIC.

From the results of the measurement shown in FIG. 3, the samples of the comparison examples containing less than 1.0 wt % (0.5 wt %) of the TAIC had gel fraction percentages at not more than 9%. It has been found that the gel fraction percentage was not more than 30 to 50 kGy irrespective of the concentrations of the TAIC and that the effect at 20 kGy was 80 to 90% of that of the effect at the gel fraction percentage of 30 to 50 kGy. It was also confirmed that as the irradiation dose increased, the gel fraction percentage decreased gradually.

In the evaluation of the expansion characteristic, gel fraction percentages of 50 to 70% is marked as ⊚; gel fraction percentages of 10 to 50% and 70 to 90% is marked as ◯; a gel fraction percentage of 10 to 6% is marked as Δ; and gel fraction percentages of 0 to 5% and 90 to 96% is marked as X.

The shape is stored by the network formed by the crosslinking. Thus when the crosslinking density is not more than 50% and particularly less than 10%, the shrink characteristic and the heat resistance are lost. On the other hand, when the crosslinking density exceeds 70% and particularly exceeds 90%, the crosslinking progresses excessively and the samples become firm and hardly deforms. Thus the expansion characteristic and the heat shrink characteristic deteriorate. Therefore it was admitted that the expansion characteristic and the heat shrink characteristic are excellent in the range of 50 to 70%.

The preferable range of the irradiation dose of electron beams was 10 kGy to 50 kGy, as described above.

This is because when the crosslinking reaction made by using the TAIC finishes at 30 to 50 kGy, only a decomposition reaction of the molecule of the polylactic acid progresses. That is, after the crosslinking reaction finishes, the network of the crosslinking is broken at many points owing to the decomposition of the molecule of the polylactic acid, and crosslinked molecules do not contribute to the storage of the shape. Thus the heat shrink characteristic decreased.

The samples, marked as ⊚ and ◯, which consists of the sheet-shaped heat-shrinkable material are expanded in a heating condition of not less than 150 to 160° C. which is the fusing temperature of the polylactic acid nor more than 180° C., with the gel fraction percentage set to 50 to 70%.

At this expanding time, the heat-shrinkable material can be expanded to not less than 2.5 times as large as the original length thereof. Thus when the heat-shrinkable material is heated to not less than 160° C. to thermally shrink it, the crosslinking is partly broken by the TAIC, and the heat-shrinkable material returns to the shape stored by the crosslinked molecules. Thus the heat-shrinkable material shrinks to not less than 40% nor more than 70%.

Further at the glass transition temperature (a little less than 60° C.) of the polylactic acid, the shrinkage factor is not more than 10%, and the gel fraction percentage is 50 to 70% to accelerate the crosslinking. Thus the heat-shrinkable material does not deform easily at the normal temperature, and the heat resistance thereof is improved. Thereby the heat-shrinkable material can be preferably used for vehicles and outdoors.

In table 3, the samples whose expansion characteristic was evaluated as ⊚ and ◯ satisfied the following three conditions:

(1) The addition amount of the TAIC was 1.0 to 3.0 wt %,

At 1.0 to 2.0 wt %, many samples were evaluated as ⊚.

(2) The irradiation dose of electron beams was 10 kGy to 50 kGy,

(3) The gel fraction percentage was 50% to 70%

### [Measurement of Heat Shrinkage Factor]

The expanded samples were heated to measure the degree of restoration to the state before it was expanded.

As the measuring method, after the expanded samples were put into a constant temperature bath and heated to a predetermined temperature, the length there in the expanded direction was measured at intervals of 10° C. higher than 40° C.

(length) shrinkage factor (%)=(length before shrink−length after shrink)/(length before shrink)×100

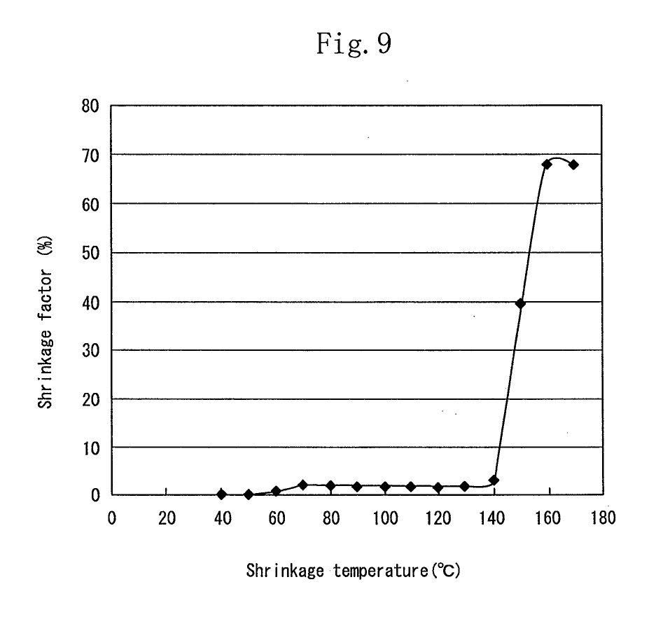

The graph of FIG. 9 shows the result of the measurement of the heat shrinkage factor of the sample which contained 1.0 wt % of the TAIC and was irradiated with electron beams in an amount of 20 kGy.

As shown in FIG. 9, irrespective of expansion percentages, the shrinkage factor was not more than 5% at temperatures not more than 140° C., and the sample started to shrink at temperatures exceeding 140° C. The shrinkage factor was about 40% at 150° C., and 65 to 70% at not less than 160° C.

Using the same polylactic acid and the same TAIC as those of the samples of the above-described examples, a heat-shrinkable tube was formed by molding the uniform mixture of the polylactic acid and the TAIC. Similarly to the above-described examples, the heat-shrinkable tube was irradiated with electron beams by changing irradiation doses. Similarly to the above-described example 1, after the heat-shrinkable tube was irradiated, it was expanded up to 2.5 times as large as the original length thereof to prepare the sample of the heat-shrinkable tube.

It could be confirmed that even in the case of the heat-shrinkable tube, not less than 1.0 wt % of the TAIC was necessary and that the gel fraction percentage could be obtained at 10 to 90% when the irradiation dose of the electron beams was 10 to 50 kGy.

As described above, the heat-shrinkable biodegradable material of the third embodiment has the crosslinked structure having the gel fraction percentage at 10 to 90% and preferably 50 to 70% owing to the irradiation of the electron beams. Therefore the biodegradable material is heat-resistant, and after the mixture is expanded, the crosslinked reticulate network shrinks because it stores the shape when the mixture of the components is thermally shrunk at the temperature used at the expanding time. Thus the biodegradable material is allowed to have a heat shrinkage factor of 40 to 80% higher than that of the conventional biodegradable material.

The fourth embodiment will be described below.

In the biodegradable material of the fourth embodiment, as the biodegradable polymer, the polysaccharide derivative such as hydrophobic starch and cellulose is used. The amount of other substances to be added to the polysaccharide derivative is not large to form the biodegradable material of the fourth embodiment having a high strength and expansion percentage. The crosslinking-type polyfunctional monomer is added to the hydrophobic polysaccharide derivative to allow the biodegradable material to have a crosslinked structure having gel fraction percentage (gel fraction dried weight/initial dried weight) at 10 to 90%.

More specifically, in the biodegradable material, 0.1 to 3 wt % of the polyfunctional monomer is added to 100 wt % of the hydrophobic polysaccharide derivative. The mixture is irradiated with the ionizing radiation in an amount of 250 kGy to generate the crosslinking by means of the polyfunctional monomer so that the hydrophobic polysaccharide derivative is crosslinked. Thereby the mixture is allowed to have the crosslinked structure having gel fraction percentage (gel fraction dried weight/initial dried weight) at 10 to 90%.

As the hydrophobic polysaccharide derivative, similarly to the second embodiment, etherified starch derivatives such as methyl starch, ethyl starch, and the like using starch such as corn starch, potato starch, sweet potato starch, wheat starch, rice starch, tapioka starch, sago starch as the material thereof; esterified starch derivatives such as acetate ester starch, aliphatic ester starch, and the like; and alkylated starch derivatives. As the hydrophobic polysaccharide derivative, it is possible to utilize derivatives similar to starch whose material is cellulose. It is also possible to utilize derivatives of other polysaccharides such as Pullulan.

These hydrophobic polysaccharide derivatives can be utilized singly or by mixing two or more kinds thereof with each other. But basically it is necessary that the substitution degree of the hydroxyl group is not less than 1.5, desirably not less than 1.8, and more desirably not less than 2.0 nor more than 3.0. That is, the derivatives are required to be sufficiently hydrophobic.

Further as additives to be added to these derivatives, to improve the flexibility thereof, plasticizers similar to those of the first through third inventions may be used.

As the polyfunctional monomer to be mixed with the hydrophobic polysaccharide derivative, the monomer having the allyl group similar to that of the first through third invention is effective. Particularly, the triallyl isocyanurate (hereinafter referred to as TAIC) and the trimethallyl isocyanurate (hereinafter referred to as TMAIC) can be preferably used.

As described above, the concentration ratio of the polyfunctional monomer to be added to the hydrophobic polysaccharide derivative is set to not less than 0.1 wt % nor more than 3 wt %. This is because the effect can be securely obtained in the range of 0.5 to 3 wt %, although the effect is admitted at 0.1 wt %.

Owing to the addition of the polyfunctional monomer to the hydrophobic polysaccharide derivative, the crosslinking reaction can be generated by irradiating the mixture thereof with the ionizing radiation. At this time, the strength can be retained to some extent by allowing the mixture to have the crosslinked structure having the gel fraction percentage (gel fraction dried weight/initial dried weight) at not less than 10%. To reliably enhance the strength, it is preferable to set the gel fraction percentage to not less than 50%.

To make the gel fraction percentage not less than 50%, it is preferable to use fatty acid ester starch, acetate ester starch, and the acetate ester cellulose or acetylated Pullulan as the hydrophobic polysaccharide derivatives, use the triallyl isocyanurate (TAIC) or the trimethallyl isocyanurate (TMAIC) as the polyfunctional monomer, and irradiate the mixture of the components with the ionizing radiation at 20 to 50 kGy.

As described above, because the polyfunctional monomer is added to the hydrophobic polysaccharide derivative such as starch or cellulose, the biodegradable material is allowed to have a crosslinking reaction by irradiating the mixture thereof with the ionizing radiation. As a result, it is possible to provide the biodegradable material with a strength in such a way that the polymer does not deform, because innumerable three-dimensional network structures are formed in the polymer. Thereby it is possible to improve the strength characteristic of the biodegradable material and allow it to have a configuration-retaining property (that is, high hardness) similar to that of the conventional general-purpose products made of the petroleum synthetic polymer material and allow the biodegradable material to be utilized as substitutions. Further by using the biodegradable material, it is possible to solve the problem of treating wastes.

In the method of the fourth embodiment for manufacturing the biodegradable material, the polyfunctional monomer is added to the hydrophobic polysaccharide derivative. After the mixture of the polyfunctional monomer and the hydrophobic polysaccharide derivative is kneaded, the mixture is molded into a predetermined shape. Thereafter the molded material is irradiated with ionizing radiation to generate a crosslinking reaction so that the biodegradable material has a crosslinked structure.

More specifically, initially the hydrophobic polysaccharide derivative is heated to a temperature at which it is softened by heating or dissolved and dispersed in a solvent capable of dissolving the hydrophobic polysaccharide derivative in acetone, ethyl acetate or the like. Thereafter the polyfunctional monomer is added to the hydrophobic polysaccharide derivative dissolved and dispersed in the solvent. Then these substances are mixed with each other as uniformly as possible. Molding may be performed in the state in which the mixture is softened by heating or in the state in which the mixture thereof is dissolved in the solvent. Alternatively the mixture may be molded into a desired shape by injection molding or the like by heating it again to soften it after it is cooled or the solvent is removed by drying it.

Similarly to the first through third inventions, as the ionizing radiation to be used for the crosslinking, γ-rays, x-rays, β-rays or α-rays can be used. In industrial production, the γ-rays emitted from cobalt 60 and electron beams emitted from an electron accelerator are preferable. The irradiation dose necessary for generating the crosslinking reaction is not less than 1 kGy nor more than 300 kGy, and favorably not less than 2 nor more than 50 kGy.

Instead of using the ionizing radiation, similarly to the first through third inventions, a chemical initiator may be used to generate the crosslinking reaction. In this case, the monomer having the allyl group and the chemical initiator are added to the hydrophobic polysaccharide derivative at a temperature not less than the melting point of the biodegradable aliphatic polyester. After the components are kneaded and mixed with each other uniformly, the temperature of the molded material composed of the mixture is increased until the chemical initiator is thermally decomposed.

Examples (examples 12 through 19) of the fourth embodiment and comparison examples (comparison examples 19 to 27) were prepared.

### EXAMPLE 12

As the hydrophobic polysaccharide derivative, fatty ester starch (CP-5 produced by Nippon Corn Starch Inc.) was used. In the derivative of the polysaccharide, the substitution degree of the hydroxyl group was about 2.0, and the substitution degree of the chain at the side of CH₂ of the fatty acid ester starch was 10 in average. The derivative of the polysaccharide was not soluble in water, but dissolvable in acetone. Thus the polysaccharide derivative is hydrophobic. After the fatty acid ester starch was melted at 150° C. by using a Lab Plast mill which is a closed kneader. Thereafter 3 wt % of the TAIC (manufactured by Nippon Kasei Inc.) which is the monomer having the allyl group was added to the fatty acid ester starch. The mixture was kneaded for 10 minutes at 20 rpm. Thereafter the mixture was thermally pressed at 150° C. to prepare a sheet having a thickness of 1 mm. In an air-removed inactive atmosphere, the sheet was irradiated with electron beams at an irradiation dose of 50 kGy by using an electron accelerator (acceleration voltage: 2 MeV, and current value: 1 mA). The obtained crosslinked material was used as the sample of the example 12.

### EXAMPLES 13, 14

Except that the addition amount of the TAIC which is the monomer having the allyl group used in the example 12 was 1 wt %, the sample of the example 13 was prepared in a manner similar to that of the example 12. Except that the addition amount of the TMAIC (produced by Nippon Kasei Inc.) which is the monomer having the allyl group was 1 wt %, the sample of the example 14 was prepared in a manner similar to that of the example 12.

### EXAMPLES 15 THROUGH 17

Except that as the hydrophobic polysaccharide derivative, acetate ester starch (CP-1 produced by Nippon Corn Starch Inc.) having a substitution degree of two was used, 1 wt % of the TAIC was used as the monomer having the allyl group, and at the kneading time and the pressing time, the heating temperature was set to 200° C. in conformity to the softening temperature of resin, the sample of the example 15 was obtained in a manner similar to that of the example 12.

As the hydrophobic polysaccharide derivative, acetate cellulose (L-30 produced by Daiseru Kagaku Inc.) having a substitution degree of two, and acetylated Pullulan (NSP-26 produced by Sanuki Kagaku Kogyo Inc.) having a substitution degree of 2.6 were used. 80 wt % of acetone and 1 wt % of the TAIC were added to 100 wt % of the polysaccharide derivative. These components were mixed with one another for five minutes by using a hybrid mixer which is a rotary kneader. After the mixture was dried, it was put into a die so that its thickness was 0.5 mm after it was dried. Thereafter it was dried slowly at the room temperature to prepare a cast film of the examples 16, 17.

### EXAMPLES 18, 19

Except that 3 wt % of HDDA was used as the polyfunctional monomer, the sample of the example 18 was prepared in a manner similar to that of example 12. Except that 3 wt % of TMPT (produced by Aldrich Inc.) was used, the sample of the example 19 was prepared in a manner similar to that of the example 12.

### COMPARISON EXAMPLES 19 THROUGH 27

The sample of each of the comparison examples 19 through 26 was prepared in a manner similar to that of the example 12, except that the samples were not irradiated with electron beams. The sample of the comparison example 27 was prepared in a manner similar to that of the example 12, except that the sample did not contain a monomer.

The difference between the examples 12 through 19 and the comparison examples 19 through 27 is shown in table 4.

TABLE 4

| | | | | Gel |
|---|---|---|---|---|
| | Hydrophobic polysaccharide | Monomer and | Irradiation dose | fraction |
| | derivative | concentration | of electron beam | percentage |
| Example | | | | |
| 12 | Fatty acid ester starch | TAIC 3% | 50 kGy | 82% |
| 13 | | TAIC 1% | | 80% |
| 14 | | TMAIC 1% | | 75% |
| 15 | Acetate ester starch | TAIC 1% | | 65% |
| 16 | Acetate ester cellulose | | | 62% |
| 17 | Acetylated Pullulan | | | 55% |
| 18 | Fatty acid ester starch | HDDA 3% | | 15% |
| 19 | | TMPT 3% | | 43% |
| Comparison | | | | |
| Example | | | | |
| 19 | Fatty acid ester starch | TAIC 3% | 0 kGy | 0% |
| 20 | | TAIC 1% | (Not irradiated) | 0% |
| 21 | | TMAIC 1% | | 0% |
| 22 | Acetate ester starch | TAIC 1% | | 0% |
| 23 | Acetate ester cellulose | | | 0% |
| 24 | Acetylated Pullulan | | | 0% |
| 25 | Fatty acid ester starch | HDDA 3% | | 0% |
| 26 | | TMPT 3% | | 0% |
| 27 | | Not used | 50 kGy | 0% |

Regarding the examples and the comparison examples, to evaluate the degree of the crosslinking of molecules performed by means of the irradiation, the gel fraction percentages were measured by the above-described method. To evaluate the effect of improving the strength of the samples by means of the crosslinking, the break strength was measured by conducting a tensile test.

The gel fraction percentage of the sample of each of the examples (when irradiated at 50 kGy) and the comparison examples is shown in table 4.

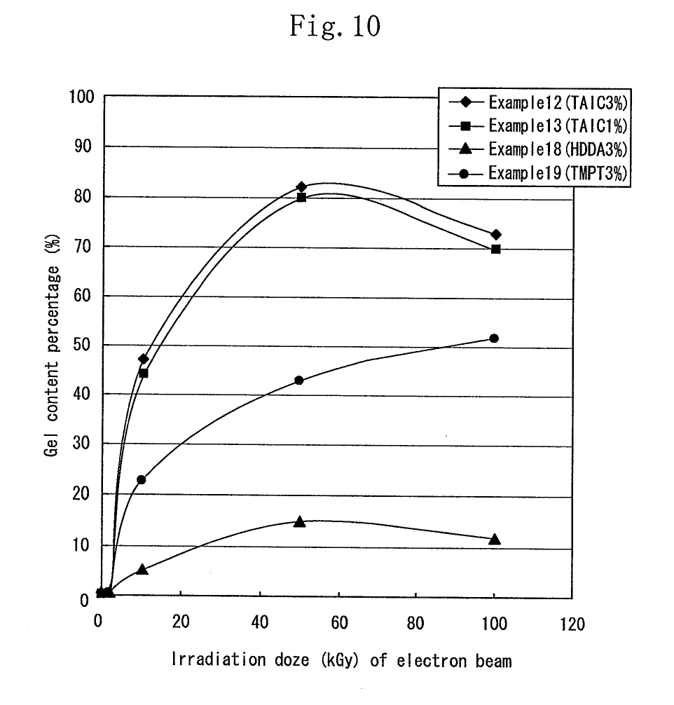

The graph showing the relationship between the irradiation dose of electron beams and the gel fraction percentage in the samples of the examples 12, 14, 18, and 19 and the comparison example 27 is shown in FIG. 10.

After samples of the example 12 and the comparison example 27 were formed as a rectangle having a width of 1 cm and a length of 10 cm, the samples were pulled at a tensile speed of 10 m/minute, with chucks spaced at 2 cm to measure the tensile break strength thereof.

break strength (kg/cm²)=tensile strength at broken time/(thickness of sample×width of sample)

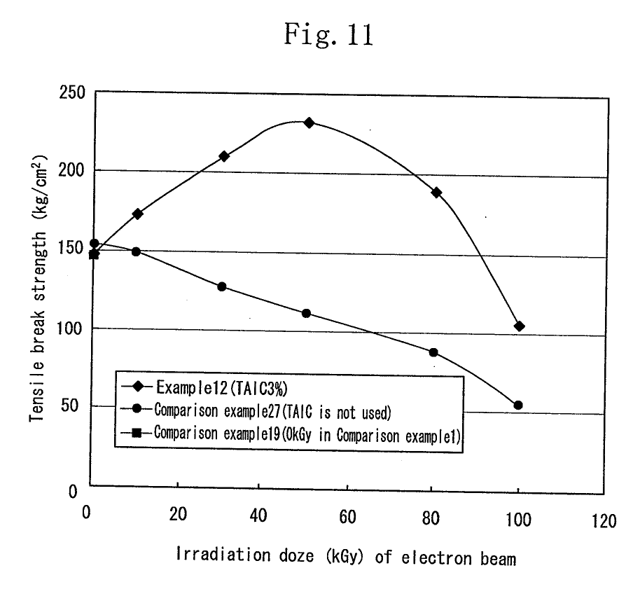

Based on the result, the graph showing the relationship between the irradiation dose of electron beams and the tensile break strength is shown in FIG. 11.

### (Evaluated Results of Examples and Comparison Examples)

From the result (table 1) of the gel fraction percentage, it has been found that unlike the samples of the comparison examples 19 through 27 not crosslinked, in the examples 12 through 19, molecules of the saccharide were crosslinked with one another by radioactive rays. It has been found that in the examples, the monomers having the allyl group such as the TAIC and the TMAIC allowed molecules to be crosslinked with one another more efficiently than the monomers such as the HDDA and the TMPT.

This is apparent from FIG. 11. The TAIC allows the crosslinking to be performed sufficiently even when the TAIC has a low concentration of 1%. Thus the TAIC is the monomer very suitable for crosslinking the hydrophobic polysaccharide derivative used as the biodegradable resin.

As shown in FIG. 11, the effect of the crosslinking is reflected in the strength thereof. That is, the sample of the example 12 which contained the TAIC added to the fatty acid ester starch to crosslink it by means of radioactive rays had a strength about twice as high as that of the sample of the comparison example 27 which did not contain the TAIC and 1.5 times as high as that of the original strength thereof in the vicinity of an irradiation dose of 50 kGy.

Considering that this crosslinking is the bonding between molecules, it is easy to estimate that the strength at high temperature, the resistance to melting-caused deformation, namely, the heat resistance have been improved. Therefore in the application in which the strength at a high temperature is necessary, it can be that the product of the present invention is effective.

As described above, the fourth invention has enabled the crosslinking of the hydrophobic polysaccharide derivative by irradiating it with the ionizing radiation. Further a low strength which is the disadvantage of the hydrophobic polysaccharide derivative can be improved greatly by the molecule-crosslinking effect. The effect of the reinforcement can be expected particularly at a high temperature from the characteristic of the reinforcing method of crosslinking molecules to each other and allows the biodegradable material of the present invention to be applied widely as the substitute of the general-purpose plastics.

1-4. (canceled)

5. A method for manufacturing a biodegradable material, the method comprising steps of:
kneading 1.2 to 3 wt % of a monomer having an allyl group and 100 wt % of a biodegradable aliphatic polyester to obtain a uniform mixture;
molding the uniform mixture into a predetermined shape; and
irradiating the molded uniform mixture with ionizing radiation to generate a crosslinking reaction so that said biodegradable aliphatic polyester is crosslinked in such a way that a gel fraction percentage of said biodegradable aliphatic polyester is not less than 75% nor more than 95%.

6. The method for manufacturing a biodegradable material according to claim 5, wherein an irradiation dose of said ionizing radiation is set to not less than 20 kGy nor more than 100 kGy.

7. A heat-resistant biodegradable material composed of biodegradable aliphatic polyester and a hydrophobic polysaccharide derivative are integrated with each other by crosslinking.

8. The biodegradable material according to claim 7, having a structure crosslinked in such a way that a gel fraction percentage (gel fraction dried weight/initial dried weight) is 50% to 95%.

9. The biodegradable material according to claim 7, wherein said hydrophobic polysaccharide derivative has a substitution degree of a hydroxyl group at not less than 2.0 nor more than 3.0; and not less than 5 wt % nor more than 30 wt % of said hydrophobic polysaccharide derivative is added to 100 wt % of said biodegradable aliphatic polyester.

10. The biodegradable material according to claim 7, wherein not less than 0.5 wt % nor more than 3 wt % of a crosslinking-type polyfunctional monomer is added to 100 wt % of said biodegradable aliphatic polyester.

11. The biodegradable material according to claim 10, wherein as said biodegradable aliphatic polyester, polylactic acid or polybutylene succinate is used;
as said hydrophobic polysaccharide derivative, acetate ester starch, fatty acid ester starch or acetate ester cellulose is used; and
as said crosslinking-type polyfunctional monomer, monomers having an allyl group such as triallyl isocyanurate, trimethallyl isocyanurate are used.

12. The biodegradable material according to claim 7, wherein a fusion molding temperature is set to a temperature range of 150° C. to 200° C. which is not less than a melting point of said biodegradable aliphatic polyester and not less than a softening point of said hydrophobic polysaccharide derivative; a tensile strength of said biodegradable material at a high temperature in the vicinity of said temperature range is 30 to 70 g/mm², and an expansion percentage of said biodegradable material is 50 to 20% so that said biodegradable material is set low in said expansion percentage and high in said tensile strength.

13. A method for manufacturing a biodegradable material according to claim 7, wherein after biodegradable aliphatic polyester, a hydrophobic polysaccharide derivative, and a crosslinking-type polyfunctional monomer are mixed with one another at a temperature not less than a melting point of said biodegradable aliphatic polyester, said mixture is molded, and thereafter said molded material is irradiated with ionizing radiation.

14. The method for manufacturing according to claim 13, wherein after 5 to 30 wt % of said hydrophobic polysaccharide derivative and 0.5 to 3 wt % of said crosslinking-type polyfunctional monomer are mixed with 100 wt % of said biodegradable aliphatic polyester, said mixture is molded, and thereafter said molded material is irradiated with ionizing radiation at 30 to 100 kGy.

15. A biodegradable material which is heat-shrinkable and composed of a mixture of biodegradable aliphatic polyester and a low-concentration monomer having an allyl group, wherein in a state in which said mixture is crosslinked by irradiating said mixture with ionizing radiation or adding a chemical initiator to said mixture, said mixture is expanded with heat being applied thereto; and wherein when said mixture is heated at a temperature not less than a temperature used at an expanding time, a shrinkage factor of said mixture is not less than 40% nor more than 80%.

16. The biodegradable material according to claim 15, wherein polylactic acid is used as said biodegradable aliphatic polyester; a gel fraction percentage (gel fraction dried weight/initial dried weight) thereof is not less than 10% nor more than 90%; a shrinkage factor at not more than 140° C. is less than 10%, and said shrinkage factor at not less than 160° C. is not less than 40% nor more than 80%.

17. A method for manufacturing a biodegradable material according to claim 15, wherein a crosslinking-type polyfunctional monomer is added at a low concentration to a biodegradable material and a mixture of said crosslinking-type polyfunctional monomer and said biodegradable material is kneaded, and said mixture is molded into a predetermined shape;
said mixture is irradiated with ionizing radiation to generate a crosslinking reaction so that a gel fraction percentage thereof is set to not less than 10% nor more than 90%; and
said mixture is expanded while said mixture is being heated at a temperature not less than a fusing temperature of said biodegradable material nor more than a temperature obtained by an addition of said fusing temperature and 20° C. after said mixture is irradiated with said ionizing radiation to form said mixture as a heat-shrinkable material,
wherein when said heat-shrinkable material is heated at a temperature not less than a temperature used at an expanding time, said heat-shrinkable material shrinks at a shrinkage factor in a range of not less than 40% nor more than 80%.

18. The method for manufacturing a biodegradable material according to claim 17, wherein a monomer having an allyl group is added at a low concentration to said biodegradable aliphatic polyester, and a mixture of said crosslinking-type polyfunctional monomer and said biodegradable aliphatic polyester is kneaded, and said mixture is molded into a predetermined shape;
said mixture is irradiated with ionizing radiation at not less than 1 kGy nor more than 150 kGy to generate a crosslinking reaction so that said mixture has a crosslinked structure and a gel fraction percentage (gel fraction dried weight/initial dried weight) thereof is not less than 10% nor more than 90%;
said mixture is expanded while said mixture is being heated in a range of 60° C. to 200° C. after said mixture is irradiated with said ionizing radiation to form a heat-shrinkable material,
wherein said heat-shrinkable material shrinks at a shrinkage factor in a range of not less than 40% nor more than 80% when said heat-shrinkable material is heated at a temperature not less than a temperature used when said heat-shrinkable material is expanded.

19. The method for manufacturing a biodegradable material according to claim 18, wherein polylactic acid is used as said biodegradable aliphatic polyester, and not less than 0.7 nor more than 3.0 wt % of said monomer having said allyl group is added to 100 wt % of said polylactic acid, and said polylactic acid and said monomer having said allyl group are kneaded;
said mixture is molded into a thin film, a thick sheet or a tube, and thereafter said thin film, said thick sheet or said tube is irradiated with ionizing radiation at not less than 5 kGy nor more than 50 kGy to generate a crosslinking reaction so that said thin film, said thick sheet or said tube has a crosslinked structure and a gel fraction percentage thereof is set to not less than 50% nor more than 70%; and
after said crosslinked structure is formed, said thin film, said thick sheet or said tube is heated at not less than 150° C. nor more than 180° C. to expand said thin film, said thick sheet or said tube at an expanding magnification of two to five.

20. The method for manufacturing a biodegradable heat-shrinkable material according to claim 19, wherein triallyl isocyanurate is used as said monomer having said allyl group; an addition amount of said triallyl isocyanurate is set to not less than 0.7 wt % nor more than 2.0 wt % for 100 wt % of polylactic acid; after said mixture is molded, said mixture is irradiated with electron beams at not less than 10 kGy nor more than 30 kGy; and said mixture is heated at not less than 160° C. nor more than 180° C. at said expanding time.

21. A biodegradable material, wherein a crosslinking-type polyfunctional monomer is added to a hydrophobic polysaccharide derivative to allow said biodegradable material to be crosslinked in such a way that a gel fraction percentage (gel fraction dried weight/initial dried weight) is 10 to 90%.

22. The biodegradable material according to claim 21, wherein 0.1 to 3 wt % of said crosslinking-type polyfunctional monomer is added to 100 wt % of said hydrophobic polysaccharide derivative; and a mixture is irradiated with ionizing radiation to allow said biodegradable material to have a crosslinked structure.

23. The biodegradable material according to claim 21, wherein a substitution degree of a hydroxyl group of said hydrophobic polysaccharide derivative is not less than 2.0 nor more than 3.0; and said hydrophobic polysaccharide derivative consists of one or a plurality of kinds of substances selected from among a starch derivative, cellulose derivative or Pullulan modified by etherified, esterified, alkylated or acetylated.

24. The biodegradable material according to claim 21, wherein said hydrophobic polysaccharide derivative consists of fatty acid ester starch, acetate ester starch, acetate ester cellulose or acetylated Pullulan;
said polyfunctional monomer consists of triallyl isocyanurate (TAIC) or trimethallyl isocyanurate (TMAIC); and
a gel fraction percentage is not less than 55%.

25. The biodegradable material according to claim 21, wherein said crosslinking-type polyfunctional monomer consists of a monomer having an allyl group selected from among triallyl isocyanurate (TAIC), trimethallyl isocyanurate (TMAIC), triallyl cyanurate (TAC), trimethallyl cyanurate (TMAC); and
an acrylic monomer and a methacrylic monomer selected from among 1,6-hexanediol diacrylate (HDDA) and trimethylolpropane trimethacrylate (TMPT).

26. A method for manufacturing a biodegradable material according to claim 21, wherein a crosslinking-type polyfunctional monomer is added to a hydrophobic polysaccharide derivative; and said crosslinking-type polyfunctional monomer and said hydrophobic polysaccharide derivative are kneaded; and after said mixture is molded into a predetermined shape, said molded material is irradiated with ionizing radiation to generate a crosslinking reaction so that said biodegradable material has a crosslinked structure.

27. The method for manufacturing a biodegradable material according to claim 26, wherein an irradiation dose of said ionizing radiation is set to 2 to 50 kGy.
